(19) United States
(12) Patent Application Publication
(10) Pub. No.: US 2003/0208797 A1
(43) Pub. Date: Nov. 6, 2003

(54) Production of antibodies

(76) Inventors: Leo G.J. Frenken, Vlaardingen (NL); Cornelis P.E. van der Logt, Bedford (GB); Yin-Miin Teh, Bedford (GB); Martine E. Verhoeyen, Bedford (GB); Joy E. Wilkinson, Bedford (GB); Stephen A. Jobling, Bedford (GB)

Correspondence Address: MORGAN LEWIS & BOCKIUS LLP, 1111 PENNSYLVANIA AVENUE NW, WASHINGTON, DC, 20004, US

(21) Appl. No.: 09/737,476

(22) Filed: Dec. 18, 2000

(30) Foreign Application Priority Data
Dec. 19, 1999 (EP) 99310188.0

(51) Int. Cl. A01H 5/00; C12N 15/82

(52) U.S. Cl. 800/298; 800/278; 800/279

(57) ABSTRACT

A method for modifying a plant to produce antibodies or active fragments or derivatives thereof in a desired cellular compartment comprising introducing into a plant a DNA sequence encoding a heavy chain immunoglobulin or an active fragment or derivative thereof or a sequence encoding a protein functionally equivalent thereto and plants so modified are disclosed.

### FIELD OF THE INVENTION

The present invention is in the field of applied biotechnology and relates in particular to an economic way of producing antibodies, or more particularly fragments thereof, in plants.

### BACKGROUND OF THE INVENTION

The production of antibodies in microbial or plant systems can be advantageous for a number of applications.

Firstly microbial or plant sources can be used as bioreactors. In the situation the antibodies and/or their derivatives are produced on a large scale in modified organisms. The use of such ‘bioreactors’, especially plants has the advantage that scale up to produced large quantities e.g. more than 100 kg or even more the 1000 kg of antibodies is relatively easy and does not require significant investments in harvesting or processing equipment.

Alternatively antibodies and/or their derivatives can be produced in plants with the aim of reprogramming the plant metabolism or to improve defence mechanisms of said plant. The antibodies to be produced are chosen such that they target specific enzymes to modulate their activity and/or to provide the plant with protection against pathogens such as parasites and viruses.

Several attempts have been made to express antibodies or fragments thereof in micro-organisms or plants.

Evidence from the literature suggests that where it is desired to express antibodies in microbial systems, the most favourable results are obtained using smaller fragments. Whole antibodies and large antibody fragments, such as Fab fragments, are very difficult if not impossible to produce. Smaller molecules such as single chain antibody fragments (scFv) are somewhat easier. Generally, however the best resuts are obtained if even smaller molecules such as heavy chain variable fragments (HCV) are produced (see, for example, WO 94/25591 (Unilever)).

In plant host systems, both large molecules, such as complete murine antibodies and smaller murine antibody fragments such as single chain Fv (scFv) fragments are capable of being expressed.

The production of functional complete murine antibodies in plants was first reported by Hiatt et al, Nature, 342, 76-78 (1989). Subsequent reports of the functional expression of murine monoclonal antibodies in plants include Düring et al, Plant Molecular Biology, 15, 281-293, (1990) and Ma et al, European Journal or Immunology, 24, 131-138, (1994).

Transgenic tobacco plants expressing a synthetic gene encoding an antigen binding single chain Fv protein (scFv) and which produce functional scFv protein have been described by Owen et al, Biotechnology, 10, 790-794, (1992). The expression of functional (that is having antigen-binding activity) scFv protein in transgenic plants has also been described in other reports in the literature, see, for example, Tavlaoraki et al, Nature, 366, 469-472 (1993).

Given that the production of functional complete antibodies requires the correct assembly, via covalent and non-covalent interactions, of both the antibody heavy and light chains, it might have been expected that expression of smaller antibody fragments, with their less stringent assembly requirements, would be advantageous. It has however been reported that generally, in practice, better yields are achieved with plants transformed with complete murine antibodies rather than small fragments (Ma et al, Science, 268, 716-719 (1995))

Furthermore, it is the experience of the present inventors that the expression of genes encoding scFv proteins in plants is not reliably reproducible and hence such a process would not readily lend itself to large scale production. Moreover, expression of genes encoding scFv molecules in plants can have an undesirable effect on plant cell morphology.

Benevenuto et al, Plant Molecular Biology, 17, 865-874 (1991) describe attempts to express smaller isolated murine heavy chain variable domain antibody fragments in plants. Successful expression is reported but there is no indication that binding affinity is retained.

For many purposes, it is important to be able to target antibodies or fragments thereof to specific cellular compartments. For example, where maximisation of yield is desired, targeting to the ER or the apoplastic space can help by providing a less reducing, more stable environment than is present in the cytoplasm. On the other hand, where it is desired to interfere with the activity of specific enzymes, in order to engineer metabolic changes in a plant, it is necessary to ensure that the antibodies are directed to the particular compartment in which the enzyme is active.

In order to direct the expression of the desired protein to a specific cellular compartment within a plant cell, the antibody proteins are fused to specific targeting sequences (peptides) where required. Expression in the chloroplast requires an N-terminal chloroplast targeting sequence (generally termed transit peptide) which is cleaved off upon entry into the chloroplast (reviewed in Keegstra & Cline (2000) Plant Cell 11, 557-570). There is little sequence homology among these transit peptides except that they are often rich in hydroxylated amino acides (eg serine), are deficient in acidic amino acids and they can vary in length from 30 to >100 amino acids. Various transit peptides fused to heterologous proteins have been used successfully to direct the protein to the chloroplast however there are no clear rules that will guarantee correct targeting and cleavage of the fusion protein. Some groups have used an exact fusion of the transit peptide to the heterologous protein; for example the transit peptide of the small subnit of rubisco from petunia has been used this way (Fray et al (2000) Nature Biotech 17, 1017-1020). Others have used transit peptides with two amino acids of the mature protein, eg potato granule-bound starch synthase (GBSS) (Kortsee et al (1996) Plant Journal 10, 83-90), and some have used as many as 23 amino acids of the mature protein eg the small subunit of rubisco from pea (Nawrath et al (1994) Proc. Natl. Acad. Sci USA 91, 12760-12764). Another group used the transit peptide of the Arabidopsis small subunit of rubisco plus 24 amino acids before the cleavage site and an additional two amino acids of the mature rubisco protein (Wong et al (1992) Plant Mol. Biol. 20, 81-93).

Proteins that enter the secretory system have N-terminal targeting peptides generally known as “signal peptides or sequences” which direct them initially to the endoplasmic reticulum (ER) whence, depending on additional sequences, the proteins are sorted either to vacuoles or are secreted from the cell. In addition, proteins which enter the ER and have a carboxy terminal peptide KDEL are retained within the ER membrane system. For location in the cytoplasm, no targeting sequence is required.

The targeting of classical conventional antibodies (ie IgG, scFv etc) to various cellular compartments of plants has been reviewed in Conrad and Fielder (1998) Plant Molecular Biology 38, 1010-109. Most commonly the homologous signal sequence of the murine IgG (e.g. CEA66E3) has been used to direct expression of the antibody via the secretory pathway, although the signal sequences from plants (PR1a, legumin B4, 2S storage protein) and even bacteria (pelB) and yeast have been used successfully. Generally the highest expression levels were obtained by the use of a signal peptide and C-terminal KDEL ER retention signal. There are no reports of successful targeting of antibodies to chloroplasts using a transit peptide as the targeting sequence. However, Düring et al (1990) Plant Mol. Biol. 15, 281-293, reported that when the barley α-amylase signal peptide was fused to the light and heavy chains of a monoclonal antibody, there was some apparent aberrant localisation of antibody within the chloroplast although the majority of the labelling was in the ER. The authors has no explanation for this observation and were unsure whether import into the chloroplast had actually occurred or if there were other reasons for the specific labelling. Interestingly, no labelling could be detected in the expected location of the apoplastic space or in the golgi apparatus or secretory vesicles.

WO 94/4678 (Casterman et al) describes the isolation of immunoglobulins from Camelids. These immunoglobulins hereinafter “heavy chain immunglobulins” have a characteristic distinct structure in that they are naturally devoid of light chains, with the antigen-binding sites being contained entirely within the heavy chain variable domains. This in turn leads to a characteristic structure for the heavy chains of these immunoglobulins, because the variable domains has no interaction sites for a light chain and a complete antigen-binding site has to be formed with no contribution from the hypervariable loops of a light chain variable domain. The heavy chain variable fragments (HCV) of these immunoglobulins are often referred to as VHH fragments. Such immunoglobulins or fragment thereof, show the functional properties of conventional, four chain, immunoglobulins but by virtue of their simplified structure offer advantages in preparation and use.

There are no reports in the literature of correctly targeted expression in plants of heavy chain immunoglobulins or fragments thereof. A report has appeared in a PhD thesis (Vu, 1999, Vrije Universiteit, Brussels, Belgium) of expression of a heavy chain variable domain in fusion with a chloroplast targeting peptide in Nicotiana tabacum. However, targeting to the chloroplast appears to have fragment (which remained as a fusion protein) was not established.

There remains a continuing need for the development of improved methods for the production of antibodies and fragments thereof in plants, with the aim of providing methods suitable for economical large scale production for protecting plants from pathogens and for modulating their metabolism.

### SUMMARY OF THE INVENTION

Accordingly, in a first aspect the invention provides a method for modifying a plant to produce an antibody or an active fragment or derivative thereof in a desired cellular compartment, comprising introducing into a plant a DNA sequence encoding a heavy chain immunoglobulin or an active fragment or derivative thereof, or a sequence encoding a protein functionally equivalent thereto, said DNA sequence being operably linked to one or more promoters and provided, as appropriate, with an additional sequence encoding a peptide sequence capable of targeting said antibody or fragment or derivative thereof to said desired cellular compartment.

Also provided is a method for preparing a heavy chain immunoglobulin or an active fragment or derivative thereof comprising extracting said immunoglobulin or fragment or derivative thereof from a plant modified according to the first aspect of the invention.

In an alternative aspect, the invention provides the use of a DNA sequence encoding a heavy chain immunoglobulin or an active fragment or derivative thereof, or a sequence encoding a protein functionally equivalent thereto, to cause a plant to produce said antibody or fragment or derivative thereof or protein functionally equivalent thereto in a desired cellular compartment.

Also provided is a modified plant having, in a desired cellular compartment, enhanced levels of heavy chain immunoglobulins or active fragments or derivatives thereof, or proteins functonally equivalent thereto, particularly compared to equivalent but unmodified plants.

Seeds, fruits and progeny of such plants and hybrids are included within the invention.

Food products such as sauces, dressings, tomato products such ketchups, meals, juices and soups, comprising a plant or part thereof according to the invention are also provided.

Also provided are skin and hair protective compositions and pharmaceutical compositions comprising a plant or part thereof according to the invention.

As used herein, “plant” means a whole plant or part thereof, or a plant cell or group of plant cells, or an extract thereof. The invention is particularly directed at transforming whole plants and the use of the whole plant or significant parts thereof.

The term “antibody” refers to an immunoglobulin which may be derived from natural sources or synthetically produced, in whole or in part. An “antibody fragmant”, alternatively an “active fragment”, is a portion of a complete antibody or immunoglobulin which retains the ability to exhibit at least part, and preferably all, of the antigen binding activity.

A “heavy chain immunoglobulin” is an immunoglobulin naturally devoid of any variable light chain domains but which is capable of specifically combining with an antigen. The antigen-binding capacity and specificity is located exclusively in the immunoglobulin heavy chains, more specifically in the heavy chain variable domains.

The “sequence encoding the heavy chain immunoglobulin or an active fragment or derivative thereof” encompassing a genomic or cDNA clone or a sequence which in proper reading frame encodes an amino acid seqence which is functionally equivalent to the amino acid sequence of the protein encoded by the genomic or cDNA clone. By “functionally equivalent” is meant any protein or fragment or derivative thereof which has the same or simliar antigen-binding properties, said antigen-binding capacity being located in a single binding domain. It should be understood, however, that isolated VH domains of conventional antibodies are not included within the scope of the invention. Similarity in functionality can be evaluated by routine screening assays, for example, by assaying the binding affinity of the immunoglobulins produced upon expression in plants.

“functionally equivalent” derivative may be characterised by an insertion, deletion or substitution of one or more amino acid residues in the sequence of the heavy chain immunoglobulin or active fragment thereof and includes within its scope fusion molecules. Such derivative may readily be made by using conventional techniques well know in the art such as peptide synthesis techniques or recombinant DNA manipulation of a gene encoding a heavy chain immunoglobulin by site directed mutagenesis.

It will be appreciated that immunoglobulins or fragments or derivatives thereof modified to enable them to function as binding domains in the same way as immunoglobulins naturally devoid of light chain domains (“heavy chain immunoglobulins”) may also suitably be used according to the invention.

A “gene” is a DNA sequences encoding a protein, including modified or synthetic DNA sequences or naturally occurring sequences encoding a protein, and excluding the 5' sequence which drives the initiation of transcription.

“Operably linked to one or more promoters” means the gene, or DNA sequence, is positioned or connected to the promoter in such a way to ensure its functioning. The promoter is any sequence sufficient to allow the DNA to be transcribed. After the gene and promoter sequences are joined, upon activation of the promoter, the gene will be expressed.

An “equivalent, unmodified” plant is a plant which has a substantially identical genotype to a modified plant of the invention excepting the introduced sequences present in the plant of the invention.

A “construct” is a polynucleotide comprising nucleic acid sequence not normally associated in nature.

plant transformed (Example 5) with the expression vector pPV.8-GBSS-HCV33-myc (of Example 1). The positive control was at a concentration of 20 μg/ml. Total protein concentration in the plant extract was 1-2 mg/ml.

FIG. 4 shows the results of immuno-histology studies, as reported in Example 5, on a tobacco plant transformed with the expression vector pPV.8-GBSS-HCV33-myc (of Example 1), confirming that HCV33 fragment is localised in the chloroplasts.

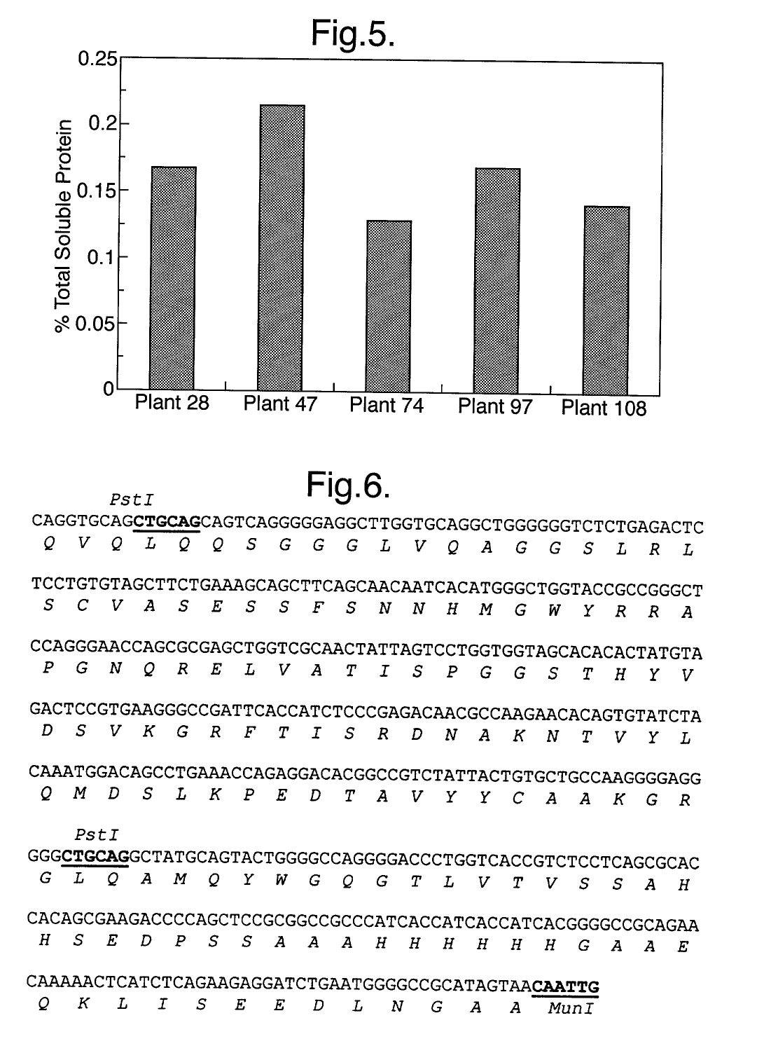

FIG. 5 shows the amount of active antibody present in fruit columella extracts of a tomato plant transformed (Example 5) with the plant expression vector pPV.8-GBSS-HCV33-myc (of Example 1), expressed as % of total soluble protein, as determined via direct binding ELISA.

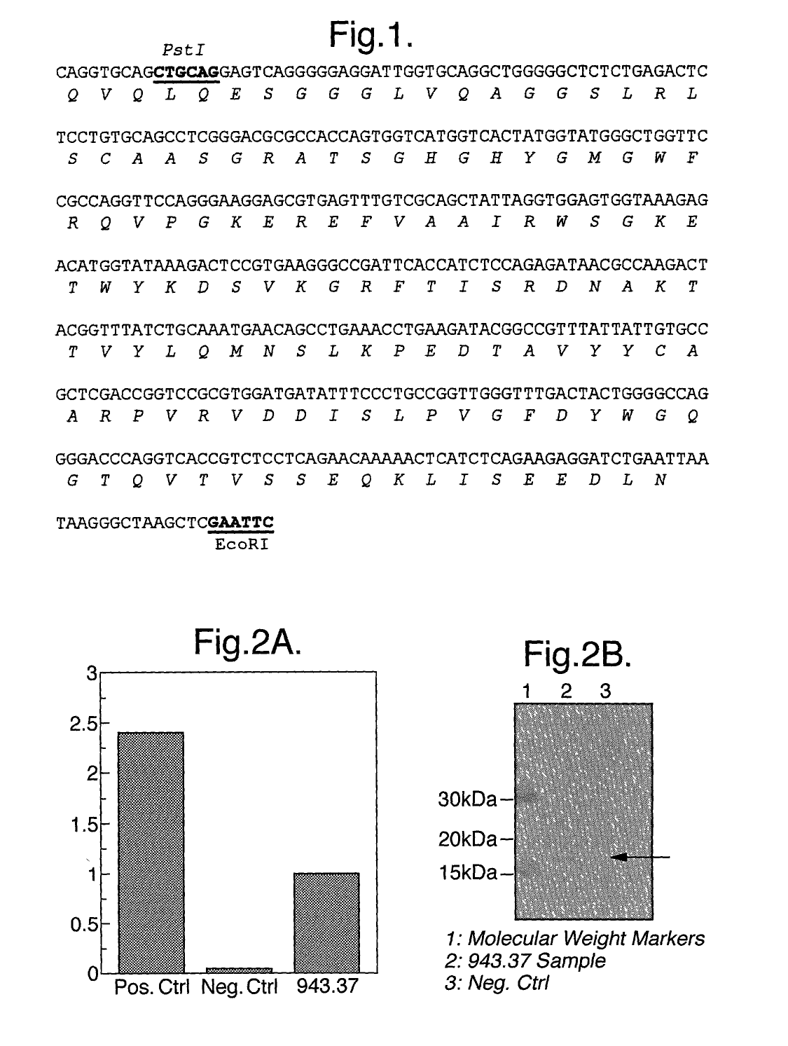

FIG. 6 shows the nucleotide sequence (SEQ. ID. No. 4) and corresponding protein sequence (SEQ. ID. No. 5) of a fragment encoding the heavy chain variable domain of an anti-potato SBEII antibody (denoted Clone1) with attached peptide linker groups denoted His6 (His-His-His-His-His-His) and myc (as described in FIG. 1). (Potato SBEII is a starch branching enzyme also denoted SBE A; Jobling et. al., Plant Journal, 18, 163-171 1999).

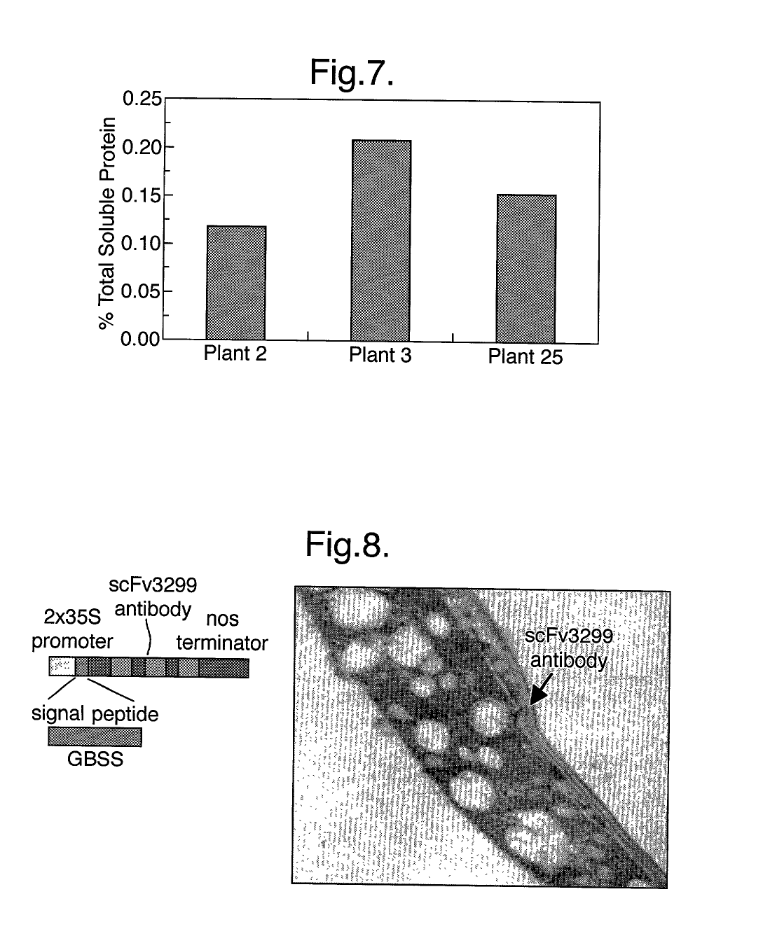

FIG. 7 shows the amount of active antibody present in whole leaf extract of a tobacco plant transformed (Example 5) with the plant expression vector pSJ35-CERV-GBSS-Clone1-His6-myc (of Example 1), expressed as % of total soluble protein, as determined via direct binding ELISA.

FIG. 8 shows the results of immuno-histology studies as reported in Example 6 on a tobacco plant transformed with the expression vector pSJ.34-2×35S-GBSS-scFv3299-hydrophilII (of Example 1). It is clear that plastid formation/function is disrupted.

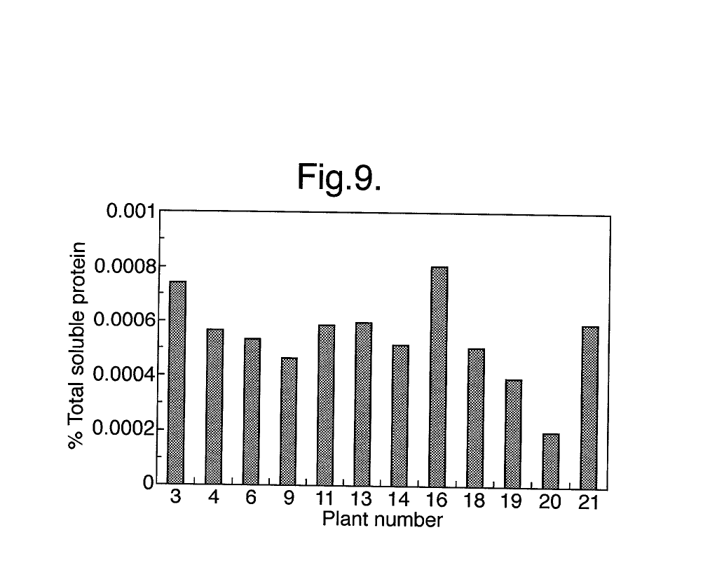

FIG. 9 shows the amount of active antibody present in leaf extract of tobacco plants transformed (Example 7) with the plant expression vector pPV.8-PR1a-HCV33-myc (of Example 1), expressed as % of total soluble protein, as determined via direct binding ELISA.

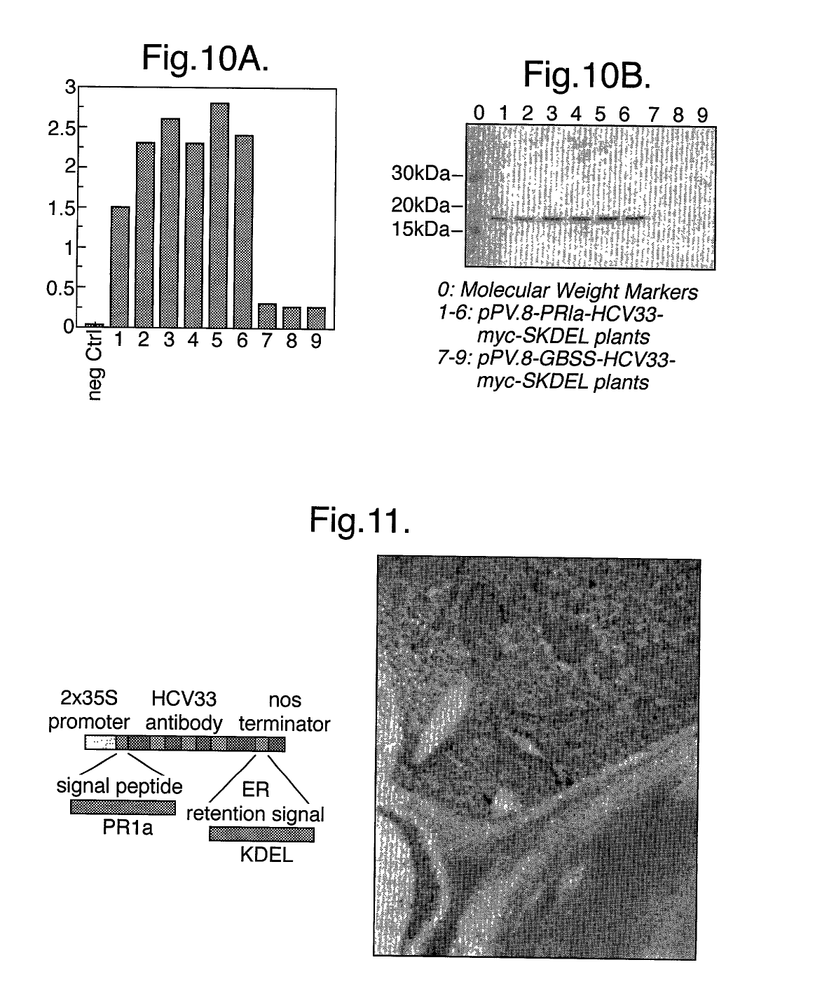

FIG. 10 shows a specificity ELISA assay (A) and SDS-PAGE/western blot analysis (B) of whole cell extracts of tobacco plants transformed (Example 8) with two plant expression vectors, namely pPV.8-PRIa-HCV33-myc-SEKDEL and pPV.8-GBSS-HCV33-myc-SEKDEL (of Example 1). Total protein concentration in the plant extract was 1-2 mg/ml.

FIG. 11 shows the results of immuno-histology studies as reported in Example 8 on a tobacco plant transformed with the expression vector pPV.8-PRIa-HCV33-myc-SEKDEL (of Example 1) confirming that the HCV33 material, directed to the secretory pathway using the PRIa leader sequence, and retained in the endoplasmic reticulum by the C-terminal tag SEKDEL.

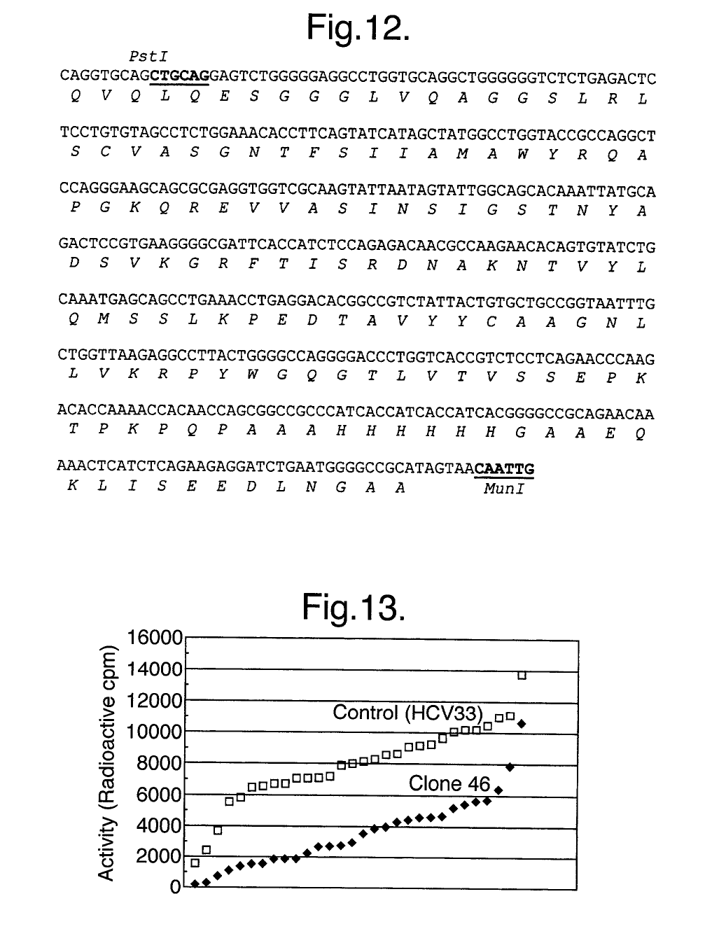

FIG. 12 shows the nucleotide sequence (SEQ. ID. No. 6) and corresponding protein sequence (SEQ. ID. No. 7) of a fragment encoding the heavy chain variable domain of an anti-potato SBEII antibody (denoted Clone46) with attached peptide linker groups denoted His6 and myc (as described in FIG. 1).

FIG. 13 shows that the SBE (starch branching enzyme) activity of whole leaf extracts of 30 tobacco plants independently transformed (Example 9) with the plant expression vector pSJ.35-CERV-GBSS-Clone46-His6-myc (of Example 1) is significantly less than that of plants transformed with pSJ.35-CERV-GBSS-HCV33-myc (of Example 1).

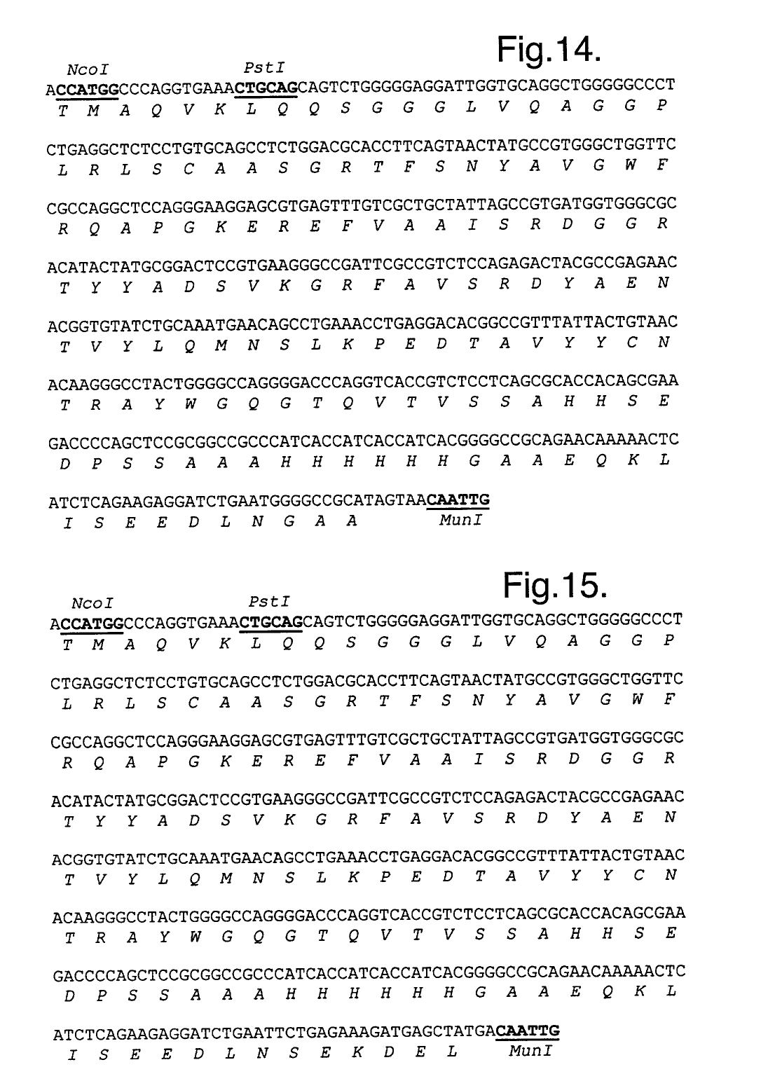

FIG. 14 shows the nucleotide sequence (SEQ. ID. No. 8) and corresponding protein sequence (SEQ. ID. No. 9) of a fragment encoding the heavy chain variable domain of an anti-GUS antibody (denoted Clone18) with attached peptide linker groups denoted His6 and myc (as described in FIG. 1). (GUS is β-glucuronidase, available from Sigma.)

FIG. 15 shows the nucleotide sequence (SEQ. ID. No. 10) and corresponding protein sequence (SEQ. ID. No. 11) of a fragment encoding the heavy chain variable domain of an anti-GUS antibody (denoted Clone18) with an endoplasmic retention signal (SEKDEL) attached peptide linker groups denoted His6 and myc (as described in FIG. 1).

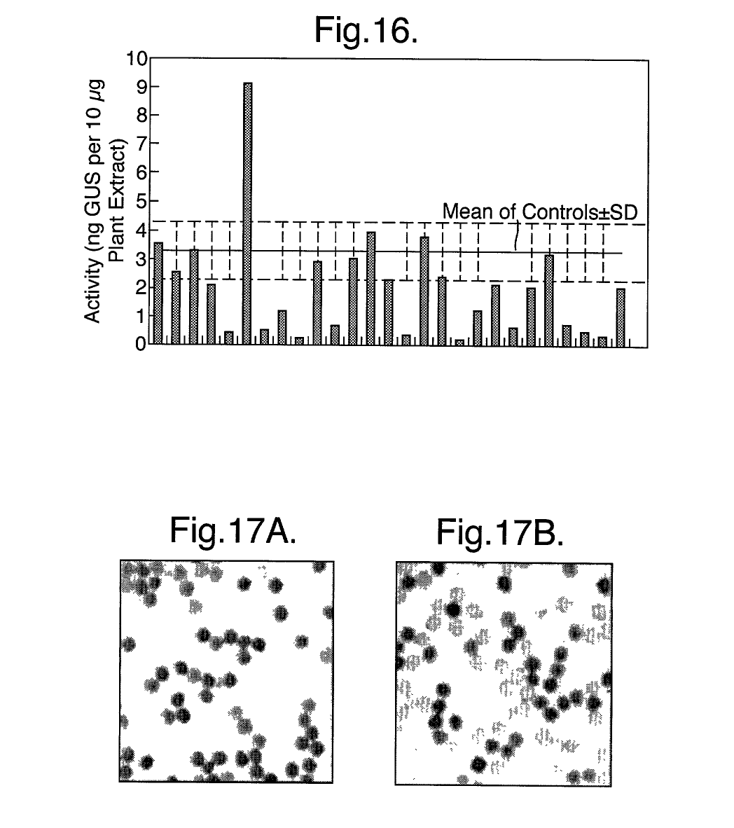

FIG. 16 shows that the GUS (β-glucuronidase) activity of whole leaf extracts of 30 pCJ.102 (of Example 1) transformed tobacco plants independently re-transformed (Example 10) with the plant expression vector pSJ.35-CERV-Clone18-His6-myc-SEKDEL (of Example 1) is significantly less than that of control pCJ.102 plants.

FIG. 17 shows (A) X-glu staining of pollen from a tobacco plant transformed (Example 11) with pSJ.35-Lat52-HCV33-myc-SEKDEL (of Example 1), and (B) X-glu staining of pollen from a tobacco plant transformed (Example 11) with pSJ.35-Lat52-Clone18-His6-myc-SEKDEL (of Example 1).

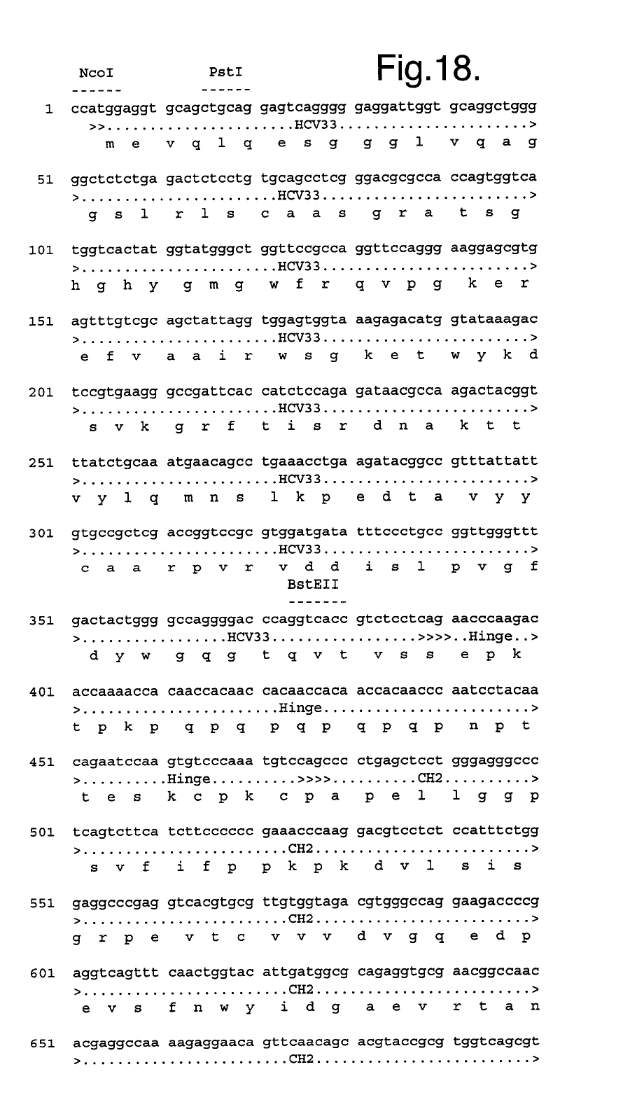

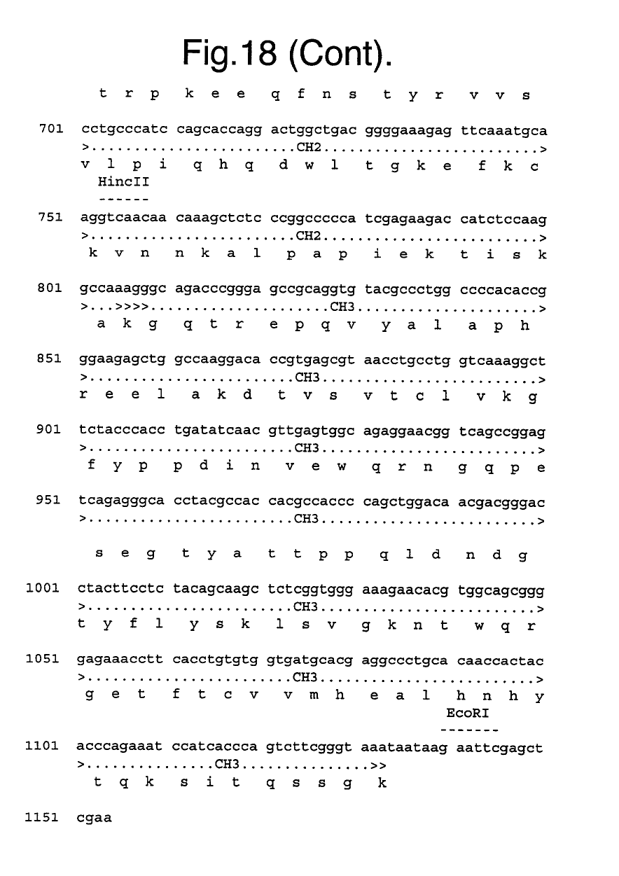

FIG. 18 shows the nucleotide sequence (SEQ. ID. No. 54) and corresponding protein sequence (SEQ. ID. No. 55) of a fragment encoding the heavy chain variable domain of an anti-RR6 antibody (denoted HCV33) linked to the non-classical heavy chain constant regions (denoted hinge-CH2-CH3).

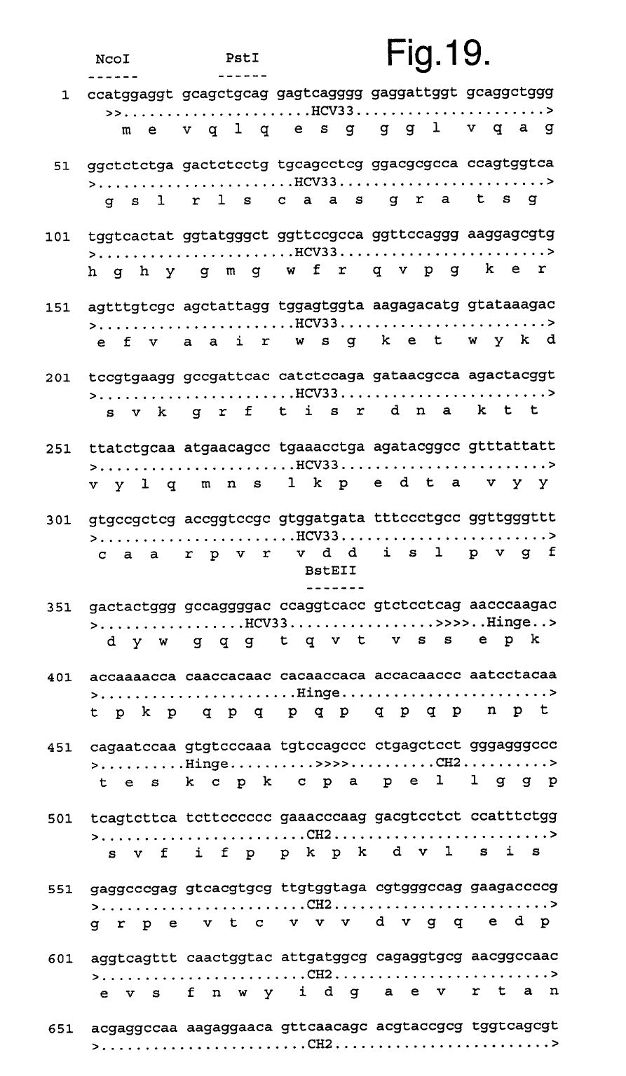

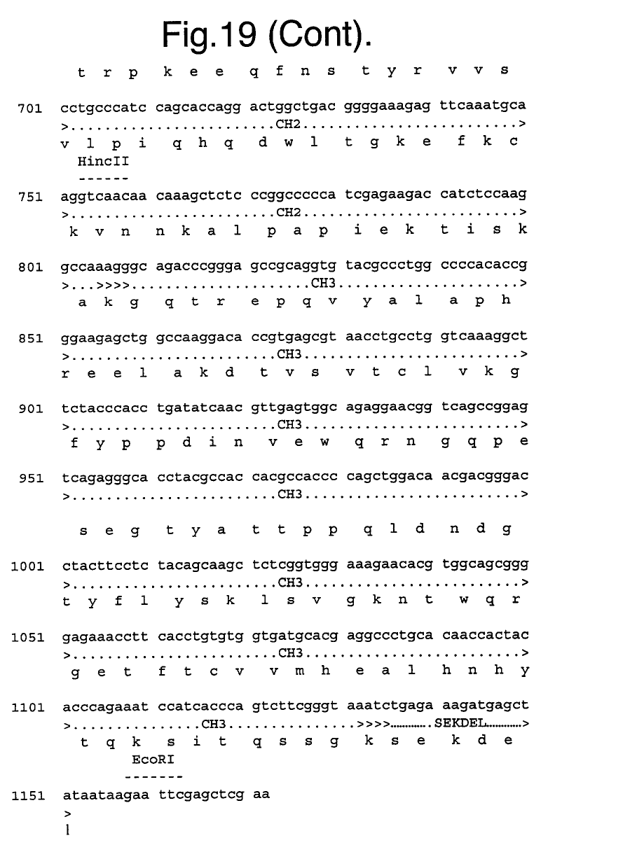

FIG. 19 shows the nucleotide sequence (SEQ. ID. No. 56) and corresponding protein sequence (SEQ. ID. No. 57) of a fragment encoding the heavy chain variable domain of an anti-RR6 antibody (denoted HCV33) linked to the non-classical heavy chain constant regions (denoted Hinge-CH2-CH3) with an endoplasmic retention signal (SEKDEL).

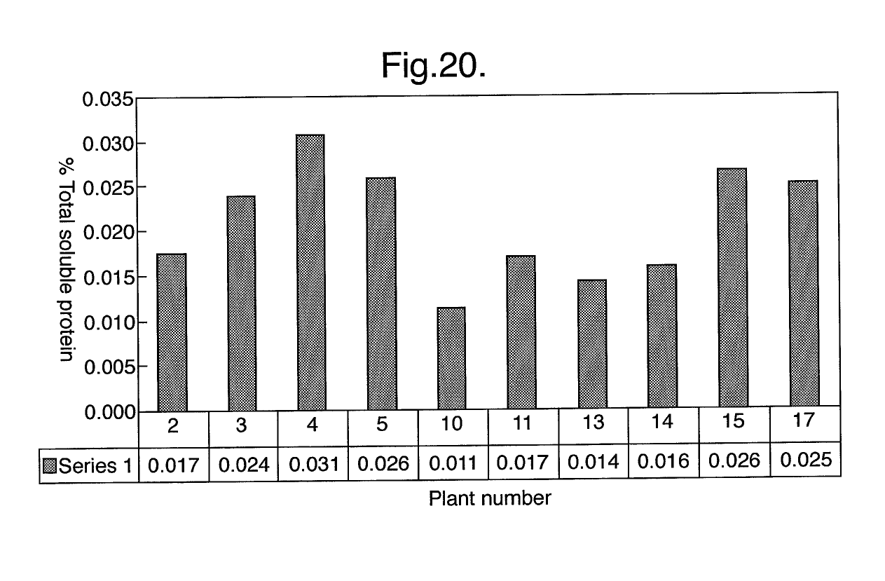

FIG. 20 shows the amount of active antibody present in whole leaf extract of a tobacco plant transformed (Example 12) with the plant expression vector pPV.8-HCV33-IgG2b (of Example 12), expressed as % of total soluble protein as determined via direct binding ELISA.

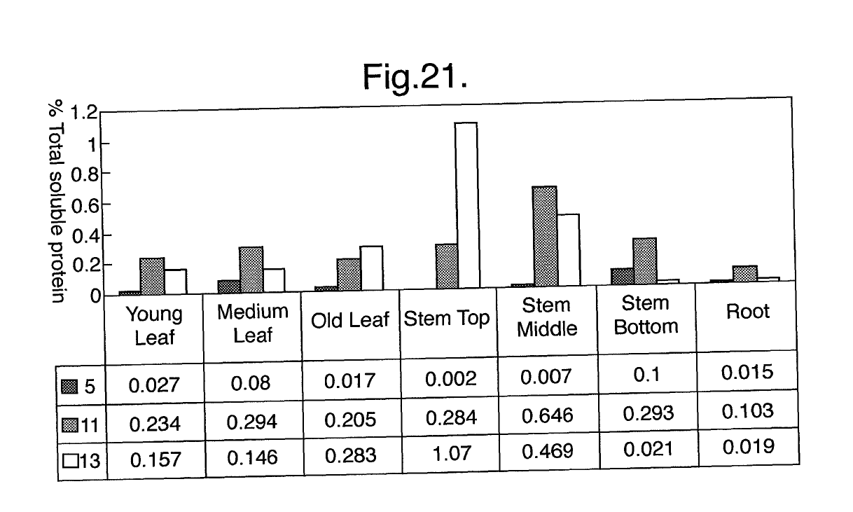

FIG. 21 shows the amount of active antibody present in whole leaf, stem and root extract of three tobacco plants (from FIG. 20) analysed 6 weeks post germination, expressed as % of total soluble protein as determined via direct binding ELISA (of Example 12).

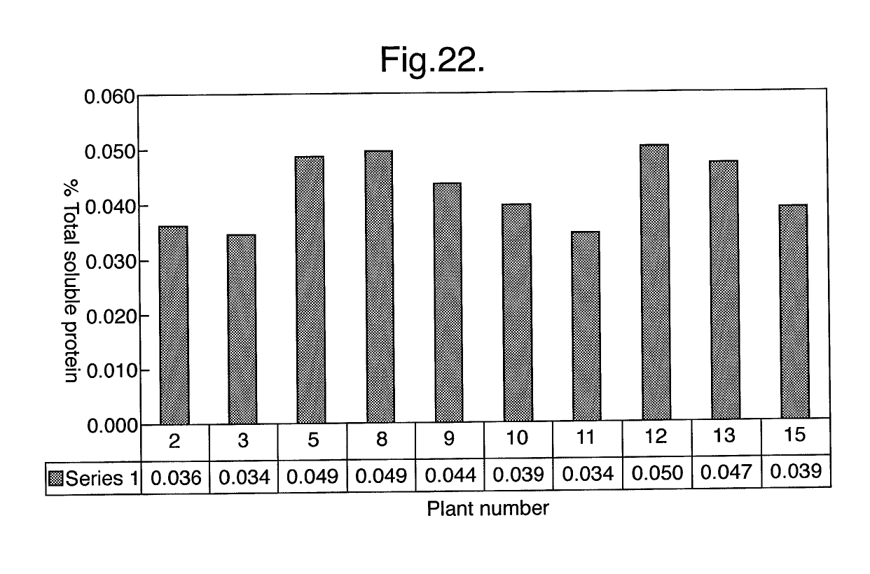

FIG. 22 shows the amount of active antibody present in whole leaf extract of a tobacco plant transformed (Example 12) with the plant expression vector pPV.8-GBSS-HCV33-IgG2b (of Example 12), expressed as % of total soluble protein as determined via direct binding ELISA.

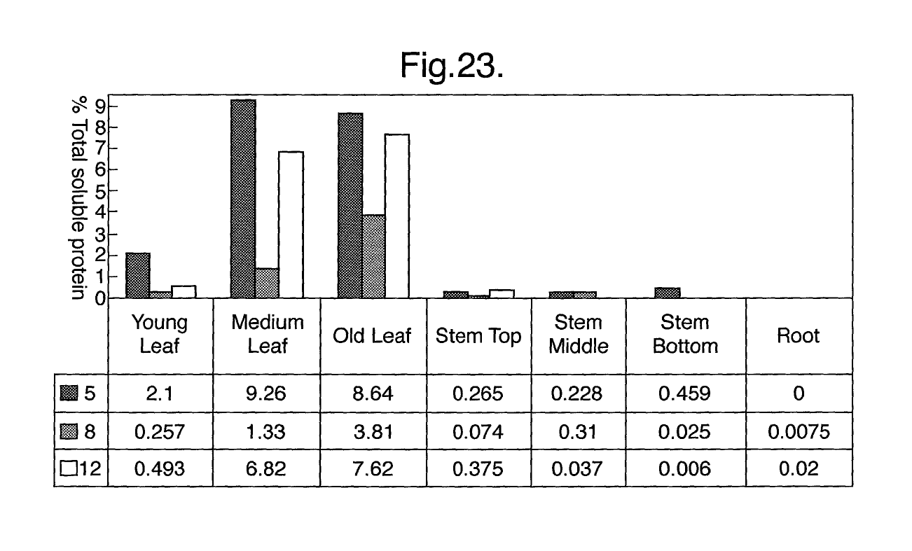

FIG. 23 shows the amount of active antibody present in whole leaf, stem and root extract of three tobacco plants (from FIG. 22) analysed 6 week weeks post germination, expressed as % of total soluble protein as determined via direct binding ELISA (of Example 12).

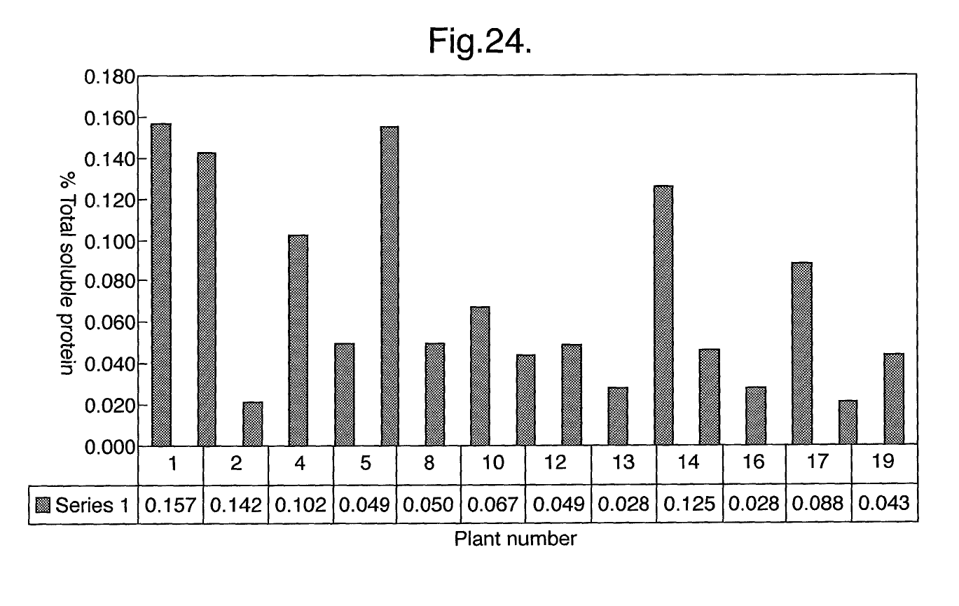

FIG. 24 shows the amount of active antibody present in whole leaf extract of a tobacco plant transformed (Example 12) with the plant expression vector pPV.8-PR1a-HCV33-IgG2b (of Example 12), expressed as % of total soluble protein as determined via direct binding ELISA.

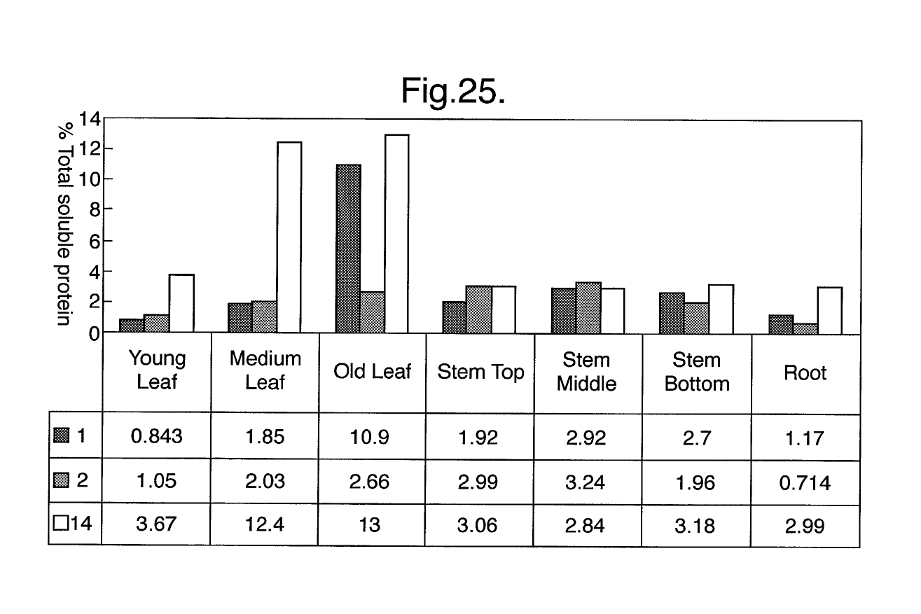

FIG. 25 shows the amount of active antibody present in whole leaf, stem and root extract of three tobacco plants (from FIG. 24) analysed 6 weeks post germination, expressed as % of total soluble protein as determined via direct binding ELISA (of Example 12).

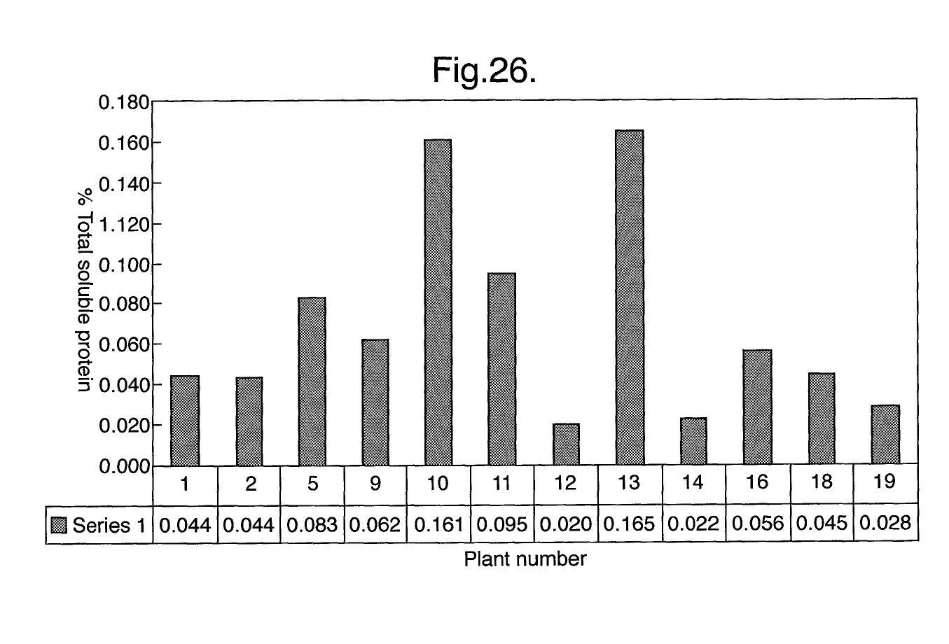

FIG. 26 shows the amount of active antibody present in whole leaf extract of a tobacco plant transformed (Example 12) with the plant expression vector pPV.8-PR1a-HCV33-IgG2b-SEKDEL (of Example 12), expressed as % of total soluble protein as determined via direct binding ELISA.

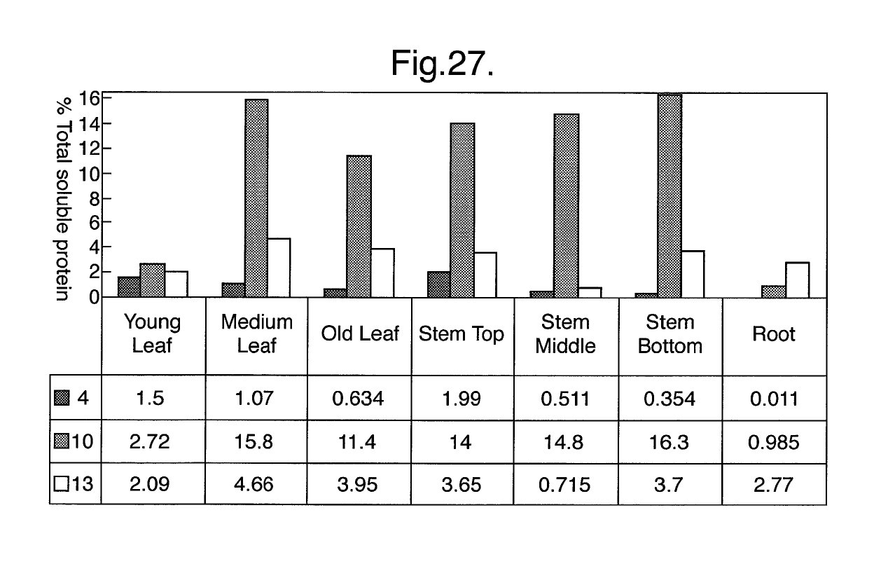

FIG. 27 shows the amount of active antibody present in whole leaf, stem and root extract of three tobacco plants (from FIG. 26) analysed 6 weeks post germination, expressed as % of total soluble protein as determined via direct binding ELISA (of Example 12).

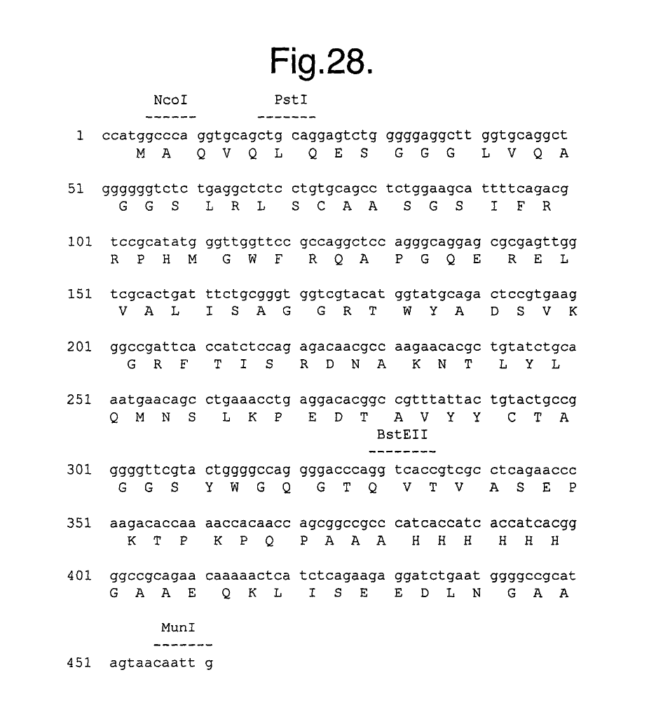

FIG. 28 shows the nucleotide sequence (SEQ. ID. No. 66) and corresponding protein sequence (SEQ. ID. No. 67) of a fragment encoding the heavy chain variable domain of an anti-potato SBEII antibody (denoted Clone68) with attached peptide linker groups denoted His6 and myc (as described in FIG. 1).

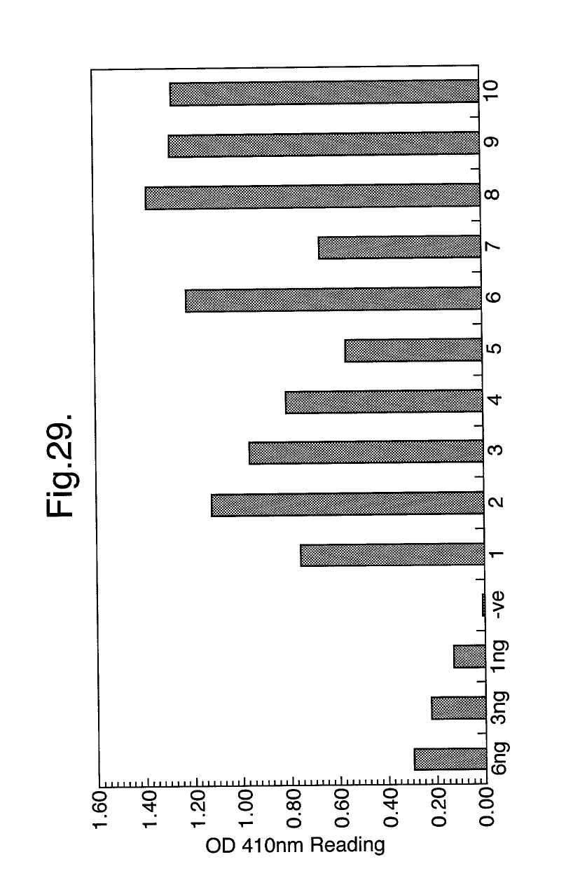

FIG. 29 shows the amount of active antibody present in whole leaf extract of a tobacco plant transformed (Example 13) with the plant expression vector pSJ35-CERV-GBSS-Clone68-His6-myc (of Example 13) expressed as OD reading as compared to the purified and quantified E. coli clone68 standard.

### DETAILED DESCRIPTION OF THE INVENTION

The present invention is based on the finding that immunologically active immunoglobulins, or active fragments or derivatives thereof, which are devoid of light chain variable domains, can advantageously be expressed at high level and successfully targeted to specific cellular locations in plant cells, with retention of binding activity and no adverse effects on plant growth or morphology.

In particular, it has been found that the method of the invention can be used to produce significant amounts of whole heavy chain immunoglobulins, with retention of binding activity, in the major compartments of the plant cell, namely plastid, cytoplasm, apoplast and endoplasmic reticulum. Selfing of transformed, antibody-expressing plants produced a new generation of plants, many of which displayed an ability to accumulate higher levels of antibody than the parent plants. The amounts of antibody were found to be markedly greater than those typically achieved when conventional antibodies are expressed in plants. The accumulation of high levels of functional antibody in the cytoplasm was a particularly unexpected result, since conventional antibodies have generally been found to be produced only in very small amounts in this location, probably owing to the problem of forming the disulphide bonds needed for structural stability in a reducing environment.

The method of the invention was similarly found to be capable of producing VHH fragments in significant amounts in these different compartments. This was the case for several different VHH fragments, in contrast to the situation with fragments based on conventional antibodies, such as scFv, where the results have been found to be very unpredictable. Importantly, it was found that the accumulation of VHH fragments in any compartment of the cell resulted in no discernible deleterious effect on the plant; again this contrasts with results obtained with scFv fragments, where expression levels were found to be unstable and accumulation of the fragment in the plastids was found to affect adversely the structure and function of these organelles.

By means of the invention, antibodies and active fragments thereof, having specificity for a target antigen may conveniently be prepared by a method which is readily amenable to industrial-scale production. Further, the ability to target the antibodies to any chosen cell compartment makes it possible to use the method of the invention to modulate the activity of specific enzymes irrespective of where in the cell the enzyme is found.

In order to modify the function of an intracellular protein, the antibody or fragment or derivative thereof must be present and active in those compartments of the host plant cell where the target protein is active. In cases where this is the cytoplasm, the reducing environment inhibits the formation of disulphide links in the antibody and so generally antibodies would not be expected to be active in the cytoplasm. Expression of classical antibodies, or fragments thereof, in the cytoplasm does not generally give rise to functional actively, for example. Similarly, expression of scFv fragments in plastids generally leads to unstable deformed plastids. The targeted expression of active antibodies or fragments or derivatives thereof in compartments of the host plant cell, especially in the cytoplasm, therefore represents a particular advantage of the method of the invention.

The present inventors have found that even when the levels of expression of heavy chain immunoglobulin or fragment or derivative thereof in the plant cell are low or undetectable by conventional protein chemical methods such as in the case of expression in the cytoplasm, a measurable effect on enzyme activity may still be achieved. The ability to modulate plant enzyme activity without accumulating antibodies in large amounts may be beneficial in some circumstances, for example, in minimising potential deleterious effects on the growth and health of the plant.

The heavy chain immunoglobulins (or fragments or derivatives thereof) to be expressed in accordance to the invention are, or are derived from, immunoglobulins that are naturally devoid of light chains, their antigen binding sites being contained entirely within the heavy chain variable domains. Especially preferred are immunoglobulins or fragments or derivatives thereof which are obtainable from Camelids as described, for example, in WO 94/4678 above. Heavy chain variable domains of immunoglobulins naturally devoid of light chains are particularly preferred for use in the present invention.

Preferred immunoglobulins for use in the invention are obtainable from Camelids especially from Lamas (for example Lama glama, Lama vicugna or Lama Paccos) or from Camelus (for example Camelus dromedaries or Camelus bactrianus).

Conveniently, a functionally equivalent protein shows at least 50% similarity to the respective amino acid sequence, preferably at least 70% similarity, more preferably at least 80%, most preferably 90-100% similarity to the respective amino acid sequence as determined by techniques well known in the art.

The invention also provides for the expression of complex derivative molecules comprising an immunoglobulin or fragment thereof as described above, associated or otherwise connected to one or more other molecules, for example, an enzyme or a further heavy chain variable domain.

The invention provides specifically for the targeting of expressed antibodies, or fragment or derivatives thereof, to specific cellular compartments. Where the desired compartment is the cytoplasm, this is the default location when no specific targeting sequence is appended to the antibody. For targeting to other compartments, sequences encoding appropriate targeting signals are fused to the sequence encoding the antibody.

Where it is desired to target an antibody to the chloroplast, a sequence encoding an appropriate transit peptide is provided, such that said peptide will be fused at the N-terminus of the expressed antibody and subsequently removed upon passage into the organelle. Any chloroplast-specific transit peptide may be used together, optionally, with a few (1-3) residues from the N-terminus of the mature protein to which said peptide is fused in nature. A number of such sequences, proven to be capable of directing successful targeting of heterologous proteins, are well known to those skilled in the art. Particularly preferred is the GBSS transit peptide sequence from potato (van der Leij et al (1991) Mol. Gen. Genet., 228, 240-248).

Where it is desired to direct secretion of an antibody into the apoplastic space, a sequence encoding an appropriate signal peptide is provided, such that said peptide will be fused at the N-terminus of the expressed antibody and subsequently removed upon passage into the endoplasmic reticulum, en route to secretion. Any signal peptide may be used, including a signal peptide that would naturally direct secretion of an antibody, or other signal peptides derived from animal, plant or microbial secreted or ER-localised proteins. Many such sequences are well known to those skilled in the art. Particularly preferred is the PRIa leader sequence (Firek et al; Plant Mol. Biol, 23 861-870 (1993)).

Where it is desired to direct an antibody to the endoplasmic reticulum, and to retain it there, a signal sequence is provided, as above, and additionally an ER-retention signal is provided. Conveniently, this takes the form of a C-terminal polypeptide extension comprising the sequence Lys-Asp-Glu-Leu (KDEL). Alternatively, the KDEL sequence may be introduced by mutation of the residues at the C-terminal end of the antibody, or fragment of derivative thereof.

It will be appreciated that the invention extends to any plant which is amenable to transformation. Suitable plants include tobacco, peas, potatoes, spinach, tomato and tea.

The DNA sequences of interest will preferably be operably linked (that is, positioned to ensure the functioning of) to one or more suitable promoters which allow the DNA to be transcribed. Suitable promoters, which may be homologous or heterologous to the gene, useful for expression in plants are well known in art, as described, for example, in Weising et al, (1988), Ann. Rev. Genetics, 22, 421-477). Promoters for use according to the invention may be inducible, constitutive or tissue-specific or have various combinations of such characteristics. Useful promoters include, but are not limited to constitutive promoters such as carnation etched ring virus (CERV), cauliflower mosaic virus (CaMV) 35S promoter, or more particularly the double enhanced cauliflower mosaic virus promoter, comprising two CaMV 35S promoters in tandem (referred to as a “Double 35S” promoter).

It may be desirable to use a tissue-specific or developmentally regulated promoter instead of a constitutive promoter in certain circumstances. A tissue-specific promoter allows for expression of immunoglobulins in certain tissues. Suitable fruit-specific promoters include the tomato E8 promoter (Deikman et al, (1988), EMBO J, 7, 3315-3320), 2A11 (Van Haaren et al, Plant Mol Biol, 21, 625-640), E4 (Cordes et al, (1989), Plant Cell, 1, 1025-1034) and PG (Bird et al, (1988), Plant Mol. Biol., 11, 651-662,) Nicholass et al, (1995), Plant Molecular Biology, 28, 423-435.

The invention provides in a further aspect an expression cassette comprising as operably linked components in the 5′-3′ direction of transcription, a promoter functional in a plant cell, one or more DNA sequences encoding a heavy chain immunoglobulin or an active fragment or derivative thereof or one or more sequences encoding a protein functionally equivalent thereto and a transcriptional and translational termination regulatory region functional in a plant cell.

The promoter and termination regulatory regions will be functional in the host plant cell and may be heterologous (that is, not naturally occurring in the host plant) or homologous (derived from the plant host species) to the plant cell and the DNA sequence. Suitable promoters which may be used are described above.

The termination regulatory region may be derived from the 3′ region of the gene from which the promoter was obtained or from another gene. Suitable termination regions which may be used are well known in the art and include Agrobacterium tumefaciens nopaline synthase terminator (Tnos), Agrobacterium tumefaciens mannopine synthase terminator (Tmas) and the CaMV 35S terminator (T35S). Particularly preferred termination regions for use according to the invention include the tobacco ribulose bisphosphate carboxylase small subunit termination region (TrbcS) or the Tnos termination region.

Such DNA constructs may suitably be screened for activity by transformation into a host plant via Agrobacterium and screening for immunoglobulin levels.

In order to be able to select for plant cells that have integrated the construct, the expression construct may conveniently be joined to a marker which permits screening or selection, according to methods well known in the art.

Conveniently, the expression cassette according to the invention may be prepared by cloning the individual promoter/gene/ terminator units into a suitable cloning vector. Suitable cloning vectors are well known in the art, including such vectors as pUC (Norrander et al, (1983, Gene 26, 101-106), pEMBL (Dente et al (1983), Nucleic Acids Research, 11, 1645-1699), pBLUESCRIPT (available from Stratagene), pGEM (available from Promega) and pBR322 (Bolivar et al, (1977), Gene, 2, 95-113). Particularly useful cloning vectors are those based on the pUC series. The cloning vector allows the DNA to be amplified or manipulated, for example by joining sequences. The cloning sites are preferably in the form of a polylinker, that is a sequence containing multiple adjacent restriction sites, so as to allow flexibility in cloning.

Suitably, the nucleotide sequences for the genes may be extracted from the Genbank nucleotide database and searched for restriction enzymes that do not cut. These restriction sites may be added to the genes by conventional methods such as incorporating these sites in PCR primers or by sub-cloning.

Preferably the DNA construct according to the invention is comprised within a vector, most suitably an expression vector adapted for expression in an appropriate host (plant) cell. It will be appreciated that any vector which is capable of producing a recombinant plant comprising the introduced DNA sequence will be sufficient.

Suitable vectors are well known to those skilled in the art and are described in general technical references such as Pouwels et al, Cloning Vectors. A laboratory manual, Elsevier, Amsterdam (1986). Particularly suitable vectors include the Ti plasmid vectors.

Transformation techniques for introducing the DNA constructs according to the invention into host cells are well known in the art and include such methods as micro-injection, using polyethylene glycol, electroporation, or high velocity ballistic penetration. A preferred method for use according to the present invention relies on agrobacterium—mediated transformation.

After transformation of the plant cells or plant, those plant cells or plants into which the desired DNA has been incorporated may be selected by such methods as antibiotic resistance, herbicide resistance, tolerance to amino-acid analogues or using phenotypic markers.

Various assays, well known in the art, may be used to determine whether the plant cell shows the presence of the desired antibody, or fragment thereof. These include binding assay such as an ELISA or radio-immunoassay.

Plant cells transformed according to the invention may be grown in an appropriate culture or cultivated in soil or some other such suitable medium by methods well known in the art. It will be appreciated that the particular procedures adopted will vary depending on the plant species used, such variations being well within the knowledge of the average skilled practitioner.

These plant cells or plants may then conveniently be harvested and the desired antibody product processed using conventional extraction and purification techniques.

Seeds produced from modified plants containing the sequence coding for the desired antibody product can subsequently be grown to generate a progeny population of modified plants from which the desired product can be isolated.

Under some circumstances, it may be desirable to retain the antibody product with the plant rather than extracting and isolating the product. Where the modified plant or part thereof concerned is edible, for example, an antibody which has an effect on ingestion can conveniently be introduced in the form of a food product. In particular, edible plants or parts thereof, modified to incorporate an antibody or fragment or derivative thereof capable of binding a pre-selected antigen may conveniently be used in a method of passively immunising an animal, preferably a mammal and in particular a human, against said antigen. Particularly suitable antigens in this case will be pathogenic organisms or derivatives or parts thereof.

In addition to well-established application in the areas of diagnosis, therapy and purification, antibodies and fragments or derivatives thereof according to the present invention may suitably by used in methods to modify the properties of the plant in which they are produced. For example, the expression in a plant of functional anti-viral antibody can be useful in reducing pathogenicity and so provide a method of producing viral resistance. Alternatively, the introduction of antibodies into plants may be used to alter or interfere with plant metabolism by modulating the function of proteins present in the plant such as plant antigens and/or block phytohormones and metabolites.

The Invention will be further illustrated by means of the following examples which are provided by way of illustration only.

Techniques used for the modification and analysis of DNA materials were performed using standard procedures well known in the art, as described, for example, in Sambrook et al, “Molecular Cloning, A Laboratory Manual, Second Edition, Cold Spring Harbor, Laboratory Press 1989 (hereinafter Sambrook), unless otherwise indicated.

Restriction sites are underlined. HCV denotes heavy chain variable domain.

### EXAMPLES

### Example 1

### Construction of HCV expression vectors

The construction of the HCV plant expression plasmids involved several cloning steps. The anti-RR6 HCV gene sequence was isolated from male llamas immunised with BSA-RR6 (as described in WO 99/23221). The sequence of the DNA fragment used in the following example (HCV33: SEQ. ID. No. 1) is given in FIG. 1.

pSJ.30

The plasmid pSJ.30 is a derivative of the binary vector pGPTV-KAN (Becker et. al., Plant Mol. Biol. 20: 1195-1197, 1992) modified as follows: An approximately 750 bp (Sac I, T4 DNA polymerase blunted - Sal I) fragment of pJIT60 (Guerineau et al., Plant Mol. Biol. 18: 815-818, 1992) containing the duplicated cauliflower mosaic virus (CaMV) 35S promoter (denoted 2×35S; Cabb-JI strain, equivalent to nucleotides 7040 to 7376 duplicated upstream of 7040 to 7433, Frank et. al., Cell 21: 285-294, 1980) was cloned into the Hind III (klenow polymerase repaired) - Sal I sites of pGPTV-KAN to create pSJ.30.

pPV.3

The shuttle vector pPV.3 was constructed by removing the HindIII/EcoRI multiple cloning site from pUC19 and replacing it by a synthetic DNA fragment, destroying the original EcoRI and HindIII sites and introducing a SgfI, HindIII, KpnI, EcoRI and XbaI restriction site. The new insert was constructed by annealing the synthetic oligonucleotides PCR.624 (SEQ. ID. No. 12) and PCR.625 (SEQ. ID. No. 13) (Table 1) yielding the insert sequence:

PCR. 624

SgfI HindIII KpnI EcoRI XbaI
5′-AGCTGCGATCGCAAGCTTGGTACCGGGAATTCTCTAGA-3′
3′-CGCTAGCGTTCGAACCATGGCCCTTAAGAGATCTTTAA-5′

PCR. 625

pPV. 5

The KpnI/EcoRI insert from pSJ.30 containing the 2×35S-promoter sequence upstream of the GUS gene, followed by the Nos terminator sequence was inserted into the KpnI/EcoRI opened pPV.3 vector yielding the intermediate vector pPV.5.

pPV.5L

The GUS coding sequence was removed from the intermediate vector pPV.5 as a SalI/SacI fragment and replaced by a synthetic DNA fragment introducing a NcoI, NheI and MunI restriction site, while leaving the original SalI/SacI sites intact. The new insert was constructed by annealing the synthetic oligonucleotides PCR.626 (Seq. ID. No. 14) and PCR.627 (Seq. ID. No. 15) (Table 1) yielding the insert sequence:

PCR. 626

SaII NcoI NheI MunI SacI
5′-TCGACCCATGGCCCGCTAGCCAATTGGAGCT-3′
3′-GGGTACCGGGCGATCGGTTAACC-5′

PCR. 627

pPV.5LN

The sequence present immediately 5′ of the start codon ATG in this vector (CCACCATGG) was replaced by the plant Kozak sequence TAAACCATGG using PCR. Oligonucleotides PCR.640 (Seq. ID. No. 16) and PCR.641 (Seq. ID. No. 17) (Table 1) was used to amplify the 189 bp 3′ fragment of the 2×35S promoter from vector pCP031 (Engelen et. al., Plant Mol. Biol., 26, 1701-1710, 1994), modifying the Kozak sequence via PCR.641. pCP031 and the amplified fragment (SEQ. ID. No. 18) were digested with HindIII/EcoRV and EcoRV/NcoI respectively and used to replace the promoter present in the intermediate vector pPV.5L. After verification of the sequence by sequencing with primer Seq. ID. No. 19 the vector pPV.5LN was used in subsequent cloning steps.

PCR. 640/PCR. 641 amplification product (SEQ. ID. NO. 18)

```
PCR. 640
CCACCCACGAGGGAACATCGTGGAAAAAGAAGACGTTCCAACCACGTCTTCAAAGCAAGT
GGTGGGTGCTCCCTTGTAGCACCTTTTTCTTCTGCAAGGTTGGTGCAGAAGTTTCGTTCA
                    EcoRV
GGATTGATGTGATATCTCCACTGACGTAAGGGATGACGCACAATCCCACTATCCTTCGCA
CCTAACTACACTATAGAGGTGACTGCATTCCCTACTGCTTGTTAGGGTGATAGGAAGCGT
                                                                                                            NcoI
AGACCCTTCCTTTATATAAGGAAGTTCATTTCATTTGGAGAGGACCTCGAGTGTAAACCA
TCTGGGAAGGAAATATATTCCTTCAAGTAAAGTAAACCTCTCCTGGAGCTCACATTTGGT
PCR. 641
TGGGAATTC
ACCCTTAAG
```

pPV.5LN-PRIa-HCV33-myc

The PstI/EcoRI HCV33-myc fragment from pPIC.HCV33-myc (described in WO 99/23221 was inserted into the NcoI/MunI opened vector pPV.5LN together with a 105 bp NcoI/PstI fragment encoding the PRIa leader sequence allowing the in-frame fusion with the 5′ framework-1 sequences of the HCV fragment.

The NcoI/PstI PRIa leader (Firek et. al., Plant Mol. Biol., 23, 861-870, 1993) sequence (including the first 4 amino acids of the HCV fragment (QVQL)) was constructed by annealing the synthetic oligonucleotides PCR.293 (Seq. ID. No. 20), PCR.294 (Seq. ID. No. 21), PCR.295 (Seq. ID. No. 22), PCR.296 (Seq. ID. No. 23), and PCR.297 (Seq. ID. No. 24) (Table 1). The product of the annealing reaction was cloned as a HindIII/PstI fragment into pUC.19. After verification of the sequence of the synthetic gene fragment by sequencing with primers Seq. ID. No. 25, 26 the NcoI/PstI fragment was excised and used in subsequent cloning reactions.

NcoI/PstI Fragment Encoding PRIA Leader Sequence (DNA Sequence Seq. ID. No. 27, Protein Sequence Seq. ID. No. 28)

```
NcoI
CCATGGCATTTGTTCTCTTTTCACAATTGCCTTCATTTCTTCTTGTCTCT
GGTACCCTAAACAAGAGAAAAGTGTTAACGGAAGTAAAGAAGAACAGAGA
    M G F V L F S Q L P S F L L V S
ACACTTCTCTTATTCCTAGTAATATCCCACTCTTGCCGTGCCCAGGTGCA
TGTGAAGAGAATAAGGATCATTATAGGGTGAGAACGGCACGGGTCCACGT
T L L L F L V I S H S C R A Q V Q
PstI
GCTGCAG
CGACGTC
    L
```

pPV.5LN-GBSS-HCV33-myc

The PstI/EcoRI HCV33-myc fragment from pPIC.HCV33-myc was inserted into the NcoI/MunI opened vector pPV.5LN together with a 252 bp NcoI/PstI fragment encoding the GBSS (including the first 4 amino acids of the HCV fragment (QVQL)) leader sequence allowing the in-frame fusion with the 5′ framework-1 sequences of the HCV fragment.

The NcoI/PstI GBSS leader sequence was constructed by amplifying potato tuber cDNA (Sp. Desiree) with synthetic oligonucleotides 132 (Seq. ID. No. 29) and 133 (Seq. ID. No. 30), 134 (Seq. ID. No. 31) and 136 (Seq. ID. No. 32) (Table 1). The products of the amplification reaction were cloned as a NcoI/HpaI, HpaI/PstI fragment into pGEM5Zf(+) (Promega). After verification of the sequence by sequencing with primers Seq. ID. No. 26, 33 (Table 1) the NcoI/PstI was excised and used in subsequent cloning reactions.

NcoI/PstI Fragment Encoding GBSS Leader Sequence (DNA Sequence Seq. ID. No. 34, Protein Sequence Seq. ID. No. 35)

```
NcoI
CCATGGCAAGCATCACAGCTTCACACCACTTTGTGTCAAGAAGCCAAACT
GGTACCGTTCGTAGTGTCGAAGTGTGGTGAAACACAGTTCTTCGGTTTGA
    M A S I T A S H H F V S R S Q T
TCACTAGACACCAAATCAACCTTGTCACAGATACGACTCAGGAACCATAC
AGTGATCTGTGGTTTAGTTGGAACAGTGTCTATCCTGAGTCCTTGGTATG
S L D T K S T L S Q I G L R N H T
TCTGACTCACAATGGTTTAAGGGCTGTTAACAAACTTGATGGGCTCCAAT
AGACTCAGTGTTACCAAATTCCCGACAATTGTTTGAACTACCCGAGGTTA
    L T H N G L R A V N K L D G L Q
CAAGAACTAATACTAAGGTAACACCCAAGATGGCATCCAGAACTGAGACC
GTTCTTGATTATGATTCCATTGTGGGTTCTACCGTAGGTCTTGACTCTGG
S R T N T K V T P K M A S R T E T
AAGAGACCTGGATGCTCAGCTACCATTGTTTGTGGAAAACAGGTGCAGCT
TTCTCTGGACCTACGAGTCGATGGTAACAAACACCTTTTGTCCACGTCGA
  K R P G C S A T I V C G K Q V Q L
    PstI
GCAG
CGTC
    Q
```

pPV.5LN-HCV33-myc

The PstI/EcoRI HCV33-myc fragment from pPIC.HCV33-myc was inserted into the NcoI/MunI opened vector pPV.5LN together with a synthetic NcoI/PstI DNA fragment allowing the in-frame fusion with the ATG start codon. The synthetic linker was constructed by annealing the synthetic oligonucleotides PCR.652 (Seq. ID. No. 36) and PCR.653 (Seq. ID. No. 37) (Table 1) yielding the insert sequence:

PCR. 652

NcoI PstI
5′-CATGCAGGTGCAGCTGCA-3′
3′-GTCCACGTCG-5′

PCR. 653

pPV.5LN-HCV33-myc-SEKDEL

The HindIII/BstEII Promoter-leader-HCV33 fragment inserts from pPV5LN-PRIa-HCV33-myc, pPV5LN-GBSS-HCV33-myc and pPV5LN-no leader-HCV33-myc was cloned into HindIII/MunI opened pPV5LN-PRIa-HCV33-myc vector together with a myc-SEKDEL encoding BstEII/EcoRI PCR fragment thus introducing the sequence SEKDEL at the C-terminal end of the myc-tag, thus yielding pPV.5LN-PRIa-HCV33-myc-SEKDEL, pPV.5LN-GBSS-HCV33-myc-SEKDEL and pPV.5LN-No leader-HCV33-myc-SEKDEL. The PCR fragment was generated using the synthetic oligonucleotides PCR.300 (Seq. ID. No. 38) and PCR.690 (Seq. ID. No. 39) (Table 1) using pPV.5LN-PRIa-HCV33-myc as a template.

pSJ.34

The BamHI restriction site of the pGPTV-KAN expression vector containing the kanamycin resistance selectable marker gene (Becker et. al., Plant Mol. Biol., 20, 1195-1197, 1992) was destroyed by restricting with BamHI followed by a Klenow reaction.

pPV.8 plant transformation vectors

The HindIII/EcoRI inserts from vectors pPV.5LN-PR1a-HCV33-myc, pPV.5LN-GBSS-HCV33-myc, pPV.5LN-No leader-HCV33-myc, pPV.5LN-PR1a-HCV33-myc-SEKDEL, pPV.5LN-GBSS-HCV33-myc-SEKDEL and pPV.5LN-No leader-HCV33-myc-SEKDEL were inserted into the HindIII/EcoRI vector fragment of pSJ.34, yielding pPV.8-GBSS-HCV33-myc, pPV.8-No leader-HCV33-myc, pPV.8-PR1a-HCV33-myc-SEKDEL, pPV.8-GBSS-HCV33-myc-SEKDEL and pPV.8-No leader-HCV33-myc-SEKDEL respectively.

pPV.5LN-2×35S-GBSS-scFv3299-hydrophilII

The PstI/EcoRI (partial) fragment containing scFv3299-hydrophilII (also known as scFv.KC in patent filing WO 96/27612 and described in more detail in WO 99/27386)was inserted into the NcoI/MunI opened vector pPV.5LN together with a 252 bp NcoI/PstI fragment encoding the GBSS leader sequence (as described for pPV.5LN-GBSS-HCV33-myc) allowing the in-frame fusion with the 5′ framework-1 sequences of the HCV fragment. By way of explanation, the sequence of scFv3299 is disclosed in W096/27612 (wherein it is described as FvKC-II), but with the BstEII-SacI linker fragment replaced with a BstEII-SacI fragment encoding a flexible linker yielding the sequence: TVTVSSGGGGSGGGGSGGGGSDIELT (Seq. ID. No. 40). The DNA sequence of the linker is described by Jackson et. al. (Selection of variants of antibodies and other protein molecules using phage display on the surface of bacteriophage fd. : in Protein engineering: A Practical Approach, eds. Rees, Sternberg and Wetzel, publ. IRL Press, Oxford, 1992)

pSJ.34-2×35S-GBSS-scFv3299-hydrophilII

The HindIII/EcoRI 2×35S promoter-GBSS-scFV3299-hydrophilII fragment of pPV.5LN-2×35S-GBSS-scFv3299-hydrophilII was inserted into the HindIII/EcoRI vector fragment of pSJ.34.

pSJ.89

A HindIII/SacI fragment containing (1×) 35S promoter-GUS.int (Vancanneyt et. al., Mol. Gen. Genet, 220, 245-250, 1990) was inserted into the HindIII/SacI vector fragment of pSJ.34. This plant transformation vector with Kan resistance contains the 35S promoter driving the GUS.int gene.

pSJ.103

The CERV promoter was isolated by PCR from infected Dianthus barbatus leaf material obtained from Dr Rene van der Lugt, Research Institute for Plant Protection (IPO-DLO), Binnehaven 5, P.O. Box 9060, NL-6700 GW, Wageningen, Netherlands, using the primers:

The approximately 380 bp fragment (corresponding to nucleotides 6737-7118 of Hull et. al., EMBO Journal, 5, 3083-3090, 1986) was amplified with oligonucleotides CERV1 (Seq. ID. No. 41) and CERV2 (14 nucleotides downstream of the transcription start site) (Seq. ID. No. 42) (Table 1) and was cloned into the TA cloning vector pT7Blue (Invitrogen) with the promoter in the same orientation as the T7 promoter. Sequence analysis showed that the isolated CERV promoter differed from the published sequence at several positions (T-C at 6790, C-T at 6826, A-G at 6872 and T-A at 6729) the most significant of which was a small deletion in a polyA tract in the 5′ untranslated leader (4A's vs. 9A's in the published sequence). These changes probably represent differences in virus isolates rather than PCR errors but these cannot be ruled out.

A HindIII-BamHI fragment from pSJ.103 was used as the source of the promoter in plant transformation vectors. Since the HindIII site was 13 nucleotides downstream of the CERV1 oligonucleotide, this sequence was not present in the promoter.

pSJ.108

The HindIII/BamHI CERV promoter fragment of pSJ.103 was inserted into the HindIII/BamHI vector fragment of pSJ.89. This plant transformation vector with Kan resistance contains the CERV promoter driving the GUS.int gene.

pPV. 7

The HindIII/EcoRI insert from pSJ.108 containing the CERV promoter sequence upstream of the GUS gene, followed by the Nos terminator sequence was inserted into the HindIII/EcoRI opened pPV.3 vector yielding the intermediate vector pPV.7.

pPV.7L

The GUS coding sequence was removed from the intermediate vector pPV.7 as a BamHI/SacI fragment and replaced by a synthetic DNA fragment introducing a NcoI, NheI and MunI restriction site, while leaving the original BamHI/SacI sites intact. The new insert was constructed by annealing the synthetic oligonucleotides PCR.628 (Seq. ID. No. 43) and PCR.629 (Seq. ID. No. 44) (Table 1) yielding the insert sequence:

PCR. 628

BamHI NcoI NheI MunI SacI
5′-GATCCCATGGCCCGCTAGCCAATTGGAGCT-3′
3′-GGTACCGGGCGATCGGTTAACC-5′

PCR. 629

pPV.7LN

The sequence present immediately 5′ of the start codon ATG in this vector (GATCCCATGG) was replaced by the plant Kozak sequence TAAACCATGG by replacing the BamHI/NheI fragment present in pPV.7L with a synthetic fragment constructed by annealing the synthetic oligonucleotides PCR.645 (Seq. ID. No. 45) and PCR.646 (Seq. ID. No. 46) (Table 1) yielding the vector pPV.7LN.

PCR. 645

BamHI NcoI NheI
5′-GATCCACCTCGAGTGTAAACCATGGCCCG-3′
3′-GTGGAGCTCACATTTGGTACCGGGCGATC-5′

PCR. 646

pPV.7LN-GBSS-HCV33-myc

The PstI/MunI HCV33-myc fragment of pPIC.HCV33-myc was inserted into the NcoI/MunI opened vector pPV.7LN together with a 252 bp NcoI/PstI fragment encoding the GBSS leader sequence (as described for pPV.5LN-GBSS-HCV33-myc) allowing the in-frame fusion with the 5′ framework-1 sequences of the HCV fragment.

pPV.7LN-GBSS-Clone1-His6-myc

The Clone1-His6-myc sequence was amplified by PCR using oligonucleotides PCR.288 (Seq. ID. No. 47) and JWHCV2 (Seq. ID. No. 48) (Table 1) from pHEN.Clone1-His6-myc. The PstI (partial)/MunI fragment was inserted into the NcoI/MunI opened vector pPV.7LN together with a 252 bp NcoI/PstI fragment encoding the GBSS leader sequence (as described for pPV.5LN-GBSS-HCV33-myc) allowing the in-frame fusion with the 5′ framework-1 sequences of the HCV fragment. The DNA sequence of clone 1 (Seq. ID. No 5, FIG. 6) was then verified with sequencing primers, Seq. ID. No. 19,49 (Table 1.

pPV.7LN-GBSS-Clone46-His6-myc

The Clone46-His6-myc sequence was amplified by PCR using oligonucleotides PCR.288 and JWHCV2 from pHEN.Clone46-His6-myc. The PstI/MunI fragment was inserted into the NcoI/MunI opened vector pPV.7LN together with a 252 bp NcoI/PstI fragment encoding the GBSS leader sequence (as described for pPV.5LN-GBSS-HCV33-myc) allowing the in-frame fusion with the 5′ framework-1 sequences of the HCV fragment. The DNA sequence of clone 46 (Seq. ID. No. 6, FIG. 12) was then verified by sequencing with primers, Seq. ID. No. 19,49 (Table 1).

pPV.7LN-Clone18-His6-myc

The Clone18-His6-myc sequence was amplified by PCR using oligonucleotides PCR.288 and JWHCV2 from pHEN.Clone18-His6-myc. The NcoI/MunI fragment was inserted into the NcoI/MunI opened vector pPV.7LN allowing the in-frame fusion with the 5′ framework-1 sequences of the HCV fragment. The DNA sequence of clone 18 (Seq. ID. No. 8, FIG. 14) was then verified by sequencing with primers, Seq. ID. No. 19,49 (Table 1).

pPV.7LN-Clone18-His6-myc-SEKDEL

The Clone18-His6-myc sequence was amplified by PCR using oligonucleotides PCR.288 and JWHCV1 (Seq. ID. No. 50) (Table 1; to generate 3′ SEKDEL endoplasmic retention signal) from pHEN.Clone18-His6-myc. The NcoI/MunI fragment was inserted into the NcoI/MunI opened vector pPV.7LN allowing the in-frame fusion with the 5′ framework-1 sequences of the HCV fragment. The DNA sequence of Clone18-His6-myc-SEKDEL (Seq. ID. No. 10, FIG. 15) was then verified by sequencing with primers, Seq. ID. No. 19,49 (Table 1).

pSJ.35

The BamHI restriction site of the pGPTV-HYG expression vector containing the hygromycin resistance selectable marker gene (Becker et. al., Plant Mol. Biol., 20, 1195-1197, 1992) was destroyed by restricting with BamHI followed by a Klenow reaction.

pSJ.35-CERV plant transformation vectors

The HindIII/EcoRI inserts from vectors pPV.7LN-GBSS-HCV33-myc, pPV.7LN-GBSS-Clone1-His6-myc, pPV.7LN-GBSS-Clone46-His6-myc, pPV.7LN-Clone18-His6-myc and pPV.7LN-Clone18-His6-myc-SEKDEL were inserted into HindIII/EcoRI vector fragment of pSJ.35, yielding pSJ.35-CERV-GBSS-HCV33-myc, pSJ.35-CERV-GBSS-Clone1-His6-myc, pSJ.35-CERV-GBSS-Clone46-His6-myc, pSJ.35-CERV-Clone18-His6-myc and pSJ.35-CERV-Clone18-His6-myc-SEKDEL respectively.

pCJ.102

Oligonucleotides 167 (Seq. ID. No. 51) and 168 (Seq. ID. No. 52) (Table 1) were used to amplify the 460bp 5′ fragment of the GUS gene (from vector pSJ.34) and modifying the 5′ end to create a NcoI site and thus modifying the second amino acid.

The PCR amplified fragment was digested with NcoI and EcoRV and inserted into HindIII/EcoRI vector fragment of pSJ.34 together with the HindIII/NcoI 2×35S promoter fragment of pPV.5LN and the EcoRV/EcoRI 3′GUS-Nos terminator fragment of pSJ.34, yielding the vector pCJ.102. The DNA sequence of the amplified fragment was verified by sequencing with primer Seq. ID. No. 53 (Table 1).

pSJ.35-Lat52-Clone18-His6-myc

The NcoI/EcoRI Clone18-His6-myc-Nos fragment of pPV.7LN-Clone18-His6-myc was inserted into the SalI/EcoRI vector fragment of pSJ.35 together with the SalI/NcoI Lat52 promoter fragment of pLAT52-7 (Twell et. al., Development, 109, 705-13, 1990).

pSJ.35-Lat52-Clone18-His6-myc-SEKDEL

The NcoI/EcoRI Clone18-His6-myc-SEKDEL-Nos fragment of pPV.7LN-Clone18-His6-myc-SEKDEL was inserted into the SalI/EcoRI vector fragment of pSJ.35 together with the SalI/NcoI Lat52 promoter fragment of pLAT52-7 (Twell et. al., Development, 109, 705-13, 1990).

pSJ.35-Lat52-HCV33-myc-SEKDEL

The PstI/SacI HCV33-myc-SEKDEL fragment of pPV.8-GBSS-HCV33-myc-SEKDEL was inserted into the SalI/SacI vector fragment of pSJ.35 with the SalI/PstI Lat52 promoter-5′ antibody gene fragment of pSJ.35-Lat52-Clone18-His6-myc.

### Example 2

### Transformation and cultivation of Nicotiana Tabacum

Cells of the agrobacterium strain LBA4404 were transformed with the described plasmids using the freeze thaw method (Plant Molecular Biology Manual PMAN A3/7).

Seeds of Nicotiana Tabacum (var. Petit Havana SR1) were surface sterilised for 10 minutes in a solution of 10% sodium hypochlorate, rinsed 3 times in sterile distilled water and planted in Murashige & Skoog (MS) basal medium+3% sucrose+0.9% agar.

After 2 weeks growth seedlings were thinned to 2 per vessel; sterile plantlets were maintained by monthly shoot cuttings onto MS basal media+3% sucrose+0.9% agar.

Discs were punched from leaves of these plants using a sterile cork borer, then incubated for 10 minutes in a culture of the agrobacterium strain LBA4404 containing the described plasmids which had been grown overnight in Lennox Broth (5 g/l NaCl, Yeast Extract 10 g/l, 10 g/l Bacto Tryptone, 15 g/l agar). The overnight culture was spun down for 10 minutes at 3000G and the growth media replaced by re-suspending the cells in MS Basal medium+3% sucrose.

Discs were removed from the culture and blotted dry on sterile filter paper before plating face down on a nurse culture plate of tobacco cells. This plate was prepared by adding 2 ml of a cell suspension culture of Nicotiana Bethiama onto a petri dish containing 25 ml of MS Salts, B5 vitamins (1 mg/l Nicotinic acid, 1 mg/lPyridoxine, 10 mg/lThiamine, 100 mg/l Inositol) +1 mg/l 2-4 D, 0.2 mg/lBAP & 0.8%agar. The cells were swirled to cover the agar then covered with a sterile filter paper disc.

Infected leaf discs were incubated at 26° C. in a light intensity of 2000 lux for 2 days, then removed from the nurse culture plates to selection media (MS Basal media+3% sucrose, 0.2mg/l IAA, 1 mg/lBAP, 0.9% agar+cefotaxime 500mg/l and kanamycin 100 mg/l or hygromycin B 20mg/l). Plates were then incubated at 3000 lux, 26 C., 16 hours day/8 hour night and transferred every 2 weeks to fresh media (as previously). Shoots that appeared from the cut edges of the discs were removed to MS Basal media+3% sucrose, cefotaxime 500 mg/l and kanamycin 100 mg/l or hygromycin B 20 mg/l to root. Leaf samples were taken from rooted shoots for testing as described in Example 3, 4, 5,7,8 & 9.

### Example 3

### Extraction and Detection of HCV Material

Extraction of Leaf Tissue for Analysis of Anti-RR6 Antibody (Denoted HCV 33)

Leaf tissue was homogenised with Polytron probe in 2 ml microcentrifuge tubes in the presence of 4 ml extraction buffer (200 mM Tris-HCl pH7.5, 5 mM EDTA, 0.1% Tween 80, 1 mM PMSF {PhenylMethylSulfonylFluoride}) per 1 gr leaf material. Solid material was removed from the homogenate by centrifugation (10 min 13,000 g). The cleared supernatant was stored at −20° C. Total protein concentration was determined using the PIERCE BCA Protein Assay Reagent detection system.

Extraction of Tomato Fruit Tissue for Analysis of Anti-RR6 Antibody (Denoted HCV33)

Fruit were dissected into peel, flesh and columella (centre of fruit), frozen and ground with a mortar and pestle in liquid nitrogen. Extracts were prepared as for leaf tissue above.

Extraction of Leaf Tissue for Analysis of Anti-Potato SBEII Antibodies (Denoted Clone1 and Clone46)

Leaf tissue was homogenised with Polytron probe in 2 ml microcentrifuge tubes in the presence of 4 ml SBE extraction buffer (100 mM Tris-HCL pH7.5, 5 mM EDTA, 2.5 mM 1,4-dithiothreitol, 0.1% (w/v) sodium metabisulphite, 5% (v/v) glycerol, 2.5% (w/v) polyvinylpolypyrollidone, 1 mM PMSF, 1 tablet Complete (Roche) per 50 ml) per 1 gr fresh weight. Solid material was removed from the homogenate by centrifugation (10 min 13,000 g). The cleared supernatant was stored at −20° C. Total protein concentration was determined using the BioRad Protein Assay Reagent detection system.

Extraction of Leaf Tissue for Analysis of Anti-GUS Antibody (Denoted Clone18)

As described for analysis of anti-RR6 antibody, except GUS extraction buffer (50 mM phosphate pH 7.0, 10 mM EDTA, 0.1% Triton X-100, 1 mM DTT).

Detection of RR6 Binding Activity

96 well ELISA plates (Greiner) were coated overnight at 4° C. with 100 μl per well of BSA-RR6 conjugate (10 μg/ml) in 0.05 M NaCO₃ buffer pH9.5. Following three washes with tap water, the plates were incubated for 1 hour at 37° C. with 250 μl per well of a blocking solution of 3% Marvel in TBST (20 mM Tris-HCL pH7.5, 140 mM Nacl, 0.1% Tween 20). Test plant extracts and appropriate standards (purified, P.pastoris produced HCV33-myc standard) in duplicate were added to wells in a total volume of 100 μl TBST, and the plates incubated at 4° C. overnight. Unbound antibody fragment was removed by 6 washes with tap water. 100 μl of the primary monoclonal anti-myc antibody No. 4111 (1 μg/mL) in 1% Marvel/TBST were added to each well. Following incubation at 37° C. for 1 hour, unbound antibody was removed by 6 washes with tap water and 100 μL of the secondary anti-mouse antibody conjugated alkaline phosphatase (1 μg/mL) was added to each well. Following incubation at 37° C. for 1 hour, unbound antibody was removed by 6 washes with tap water, and alkaline phosphatase activity was detected by adding 100 μl of 1 mg/ml pNPP in 1M diethanolamine/1 mM MgCl₂. Plates were incubated at room temperature for up to 5 hours before reading at 410 nM on a Dynex, MCX plate reader.

Detection of SBEII Binding Activity

As for RR6 binding activity, except:

100 μl/well of 5 μg/ml recombinant potato SBEII was bound to the plate (Jobling et al., Plant Journal, 18, 163-171, 1999) in PBS overnight at 4° C.

Purified and quantified clone1-myc from E.coli was used as the standard.

Blocking buffer: 1% BSA in TBST, all incubations at room temperature.

100 μl/well of 1:2,000 dilution of rabbit anti-llama IgG, as the primary detection antibody.

100 μl/well of lug/mL goat anti-rabbit antibody conjugated alkaline phosphatase, as the secondary detection antibody.

SDS-PAGE and Western Blotting

Plant extracts were analysed on 10% bis-acrylamide gel using the Bio-Rad mini-Protean II system according to manufacturers instructions. 40 μg samples and appropriate standards (purified P.pastoris produced HCV33-myc) were diluted in 50 μl 2×SDS-PAGE loading buffer (20% Glycerol, 20 mM Tris-HCL pH 8.0, 0.5% SDS, 0.005% bromophenol blue). The samples were mixed, boiled (5 minutes) and centrifuged briefly before loading onto the gel. The gels were run at 100V for 1 hour in 1× running buffer (25 mM Tris, 200 mM glycine, 1% SDS).

Proteins were transferred onto nylon membranes (Millipore) by semi dry blotting at 25V, 80 mA for 45 minutes in semi-dry blotting buffer (50 mM Tris, 40 mM glycine, 0.04% SDS, 20% methanol). The membranes were then dried overnight at room temperature. The membranes were incubated in 3% Marvel, TBST for 10 minutes then in lug/mL mouse anti-myc antibody in 1% Marvel, TBST for 60 minutes at room temperature with constant agitation. Unbound antibody was removed by washing 6 times in TBST at room temperature with constant agitation. The blot was then incubated with the secondary antibody (rabbit anti-mouse IgG conjugated to alkaline phophatase) in 1% Marvel, TBST for 60 minutes at room temperature with constant agitation. Unbound antibody was removed by washing 6 times in TBST at room temperature with constant agitation. Bound antibody was detected by incubating the filters in 10 mL of 30 mg/mL NBT and 15 mg/ml BCIP. Washing the filters with water stopped the colour reaction.

EM Studies

Step 1: Sample preparation:

Leaf samples were fixed using 1% Paraformaldehyde+0.05% glutaraldehyde in 0.05M sodium phosphate buffer pH6.8, for 4hrs at 4° C., and then washed in phosphate buffer overnight at 4° C. Samples were embedded by first dehydrating the samples through alcohol, 50, 70, 90% (15 mins) and finally absolute alcohol (2×30 mins). They were then placed in hydrophilic resin [6 parts LRGold resin+_{—}4 parts GMA (low acid)+0.1% benzoin ethyl ether (UV activator). Resin was changed several times over period of three days. Finally, samples were embedded, using freshly prepared resin, in flat bottomed plastic moulds, allowing the resin to polymerise for 24 hrs at room temperature, under nitrogen gas, using UV light (360 nm).

Step 2: Staining:

Ultrathin leaf tissue sections were collected on collodion (2% in amyl acetate) coated nickel e.m. grids. Grids were floated on 20 μl aliquots of TBS/BSA [20 mM Tris/HCL buffer pH7.6+0.23M NaCl+1 bovine serum albumin] for 20 mins at room temperature, following by incubation with 20 μl aliquots of primary antibody (anti-myc or anti-hydrophil II, 1 μg/ml) in TBS/BSA+0.05% Tween 20 for 18 hrs at room temperature. Grids were washed thoroughly using TBS. The primary antibody was detected by incubating the grids with 20 μl aliquots of secondary antibody/gold conjugate diluted in TBS/BSA (Goat a Mouse IgG; 5 nm diluted 1:200, 10 nm diluted 1:100 20 nm diluted 1:50) for 60 mins at room temperature. The grids were washed thoroughly, using TBS followed by washes with deionised water and dried. Some grids were silver enhanced, prior to poststaining with 20 aqueous Uranyl Acetate, followed by Lead Citrate. Photographs were taken using a either a Jeol 100CX Mk2 transmission electron microscope at 60,000 magnification (FIG. 4) or a Jeol 1220 transmission electron microscope at 40,000 magnification (FIG. 11).

Assay of SBE Activity

All protein samples were diluted to a concentration of 1 mg/mL using SBE extraction buffer. SBE was assayed using the phosphorylase a stimulation assay (Hawker et al., Arch. Biochem. Biophys., 160, 530-551, 1974). The reaction was carried out at 30° C. for 1 h on 50 μg protein in a final volume of 200 μl and contained 100 mM 2-(N-morpholino)ethanesulphonic acid (MES), 50 mM [U-14C]glucose-1-phosphate (370 MBq mole-1, ICN), 1.3U rabbit muscle phophorylase a (Sigma), 50 μl plant extract at 1 μmg/ml protein. Glucose polymers were precipitated and washed as described by Hawker et al., Arch. Biochem. Biophys., 160, 530-551 (1974). The final pellet was dissolved in DMSO and counted with ReadySafe-scintillation cocktail (Beckmann).

Assay of GUS Activity

All protein samples were diluted to a concentration of 1 mg/ml using GUS extraction buffer. 10 μl samples were then added to 190 μl of 1× reaction buffer (50 mM phosphate pH 7.0, 0.1% Triton X-100, 1 mM DTT, 1 mM p-nitrophenyl β-D-glucuronide) contained in the well of a flat-bottomed microtitre plate. The reaction was left to proceed at 37° C. until the wells turned visibly yellow. At this point, 80 μl of stop solution (2.5M 2-amino-2-methyl propanediol) was added and the plate read at 410 nm using a Dynex plate reader. GUS activity was calculated based on positive control wells to which known quantities of GUS were added.

Histochemical Staining for GUS Activity

Tobacco pollen was stained for GUS activity in microtitre wells for up to 1 hour in 1 mM 5-bromo-4-chloro-3-indolyl β-D-glucuronic acid according to the method of Jefferson et al., EMBO Journal 6: 3901-7, (1987). The buffer was modified by the use of histochemical staining buffer comprising 100 mM sodium phosphate pH 7.0, 10 mM EDTA, and 0.1% (v/v) Triton X-100 (Stomp, In: Editorial comments, Vol. 16, No.5. Cleveland: united State Biochemical, 1990). To minimalise spread of the stain to surrounding pollen grains, 5 mM potassium ferricyanide was added to localise staining (Lojda, Histochemie 22: 347-61, 1970; Mascarenhas and Hamilton, Plant Journal 2: 405-8, 1992).

### Example 4

### Expression of HCV Antigen Binding Activity in the Cytoplasm

Small samples of the leaves of 10 individual Tobacco plants transformed with the expression vector pPV.8-HCV33-myc were tested for the presence of anti-RR6 binding activity using ELISA. One of the tested samples showed low levels of RR6 binding activity. Detailed analysis of the anti-RR6 binding activity present in this clone (No. 943.37) using SDS-PAGE/Western blot analysis, ELISA and total protein concentration measurement showed that HCV33-myc was expressed as 0.001-0.002% of total protein present (FIG. 2 and Table 2).

### Example 5

### Expression of HCV Antigen Binding Activity in Plastids

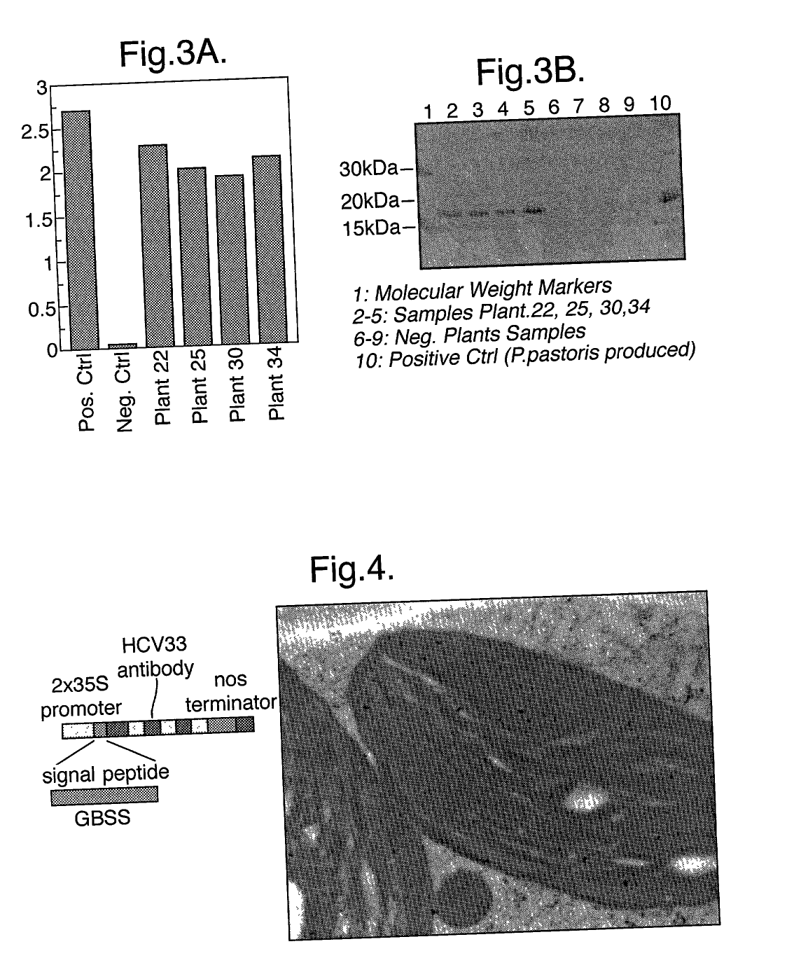

Small samples of the leaves of 15 individual Tobacco plants transformed with the expression vector pPV.8-GBSS-HCV33-myc were tested for the presence of anti-RR6 binding activity using ELISA. Seven of the tested samples showed high levels of RR6 binding activity. Detailed analysis of the anti-RR6 binding activity present in these clones using SDS-PAGE/Western blot analysis, ELISA and total protein concentration measurement showed that HCV33-myc was expressed as 0.5-1.0% of total soluble protein present (FIG. 3 and Table 2). From the Western blot it can be concluded that the construct is processed correctly and that the GBSS leader is removed from the HCV fragment. Localisation of the HCV33 material in the chloroplasts was confirmed via immuno-histology studies (FIG. 4).

The fruit of 10 individual tomato plants transformed with the expression vector pPV.8-GBSS-HCV33-myc were picked 15 days post-breaker (red stage), dissected and tested for the presence of anti-RR6 binding activity using ELISA. 5 of tested fruit expressed levels of RR6-binding activity which, by comparison to a standard curve of purified HCV33 binding to RR6, were over 0.1% (one was over 0.2%) of total soluble protein (FIG. 5 and Table 2).

Similar high levels of expression were obtained with an HCV antibody of different specificity. Small samples of the leaves of 30 individual tobacco plants transformed with the expression vector pSJ.35-CERV-GBSS-Clone1-His6-myc were tested for the presence of anti-SBEII binding activity using ELISA. 3 of the tested plants expressed levels of SBEII-binding activity over 0.1% total soluble protein (FIG. 7 and Table 2).

### Example 6

### Disruption of Plastid by scFv Expression

Tobacco plants were transformed with expression vector pSJ34-2×35S-GBSS-scFv3299-hydrophilII. After initial detection of high expression of hCG binding activity, these levels were lost×expression was unstable (not shown). Furthermore, immuno-histology studies (FIG. 8) suggested that scFv expression had an adverse affect on plastid structure and function, perhaps due to the aggregation of multiple scFv molecules at the plastid membrane.

### Example 7

### Expression of HCV Antigen Binding Activity in the Apoplastic Space

Small samples of the leaves of 21 individual Tobacco plants transformed with the expression vector pPV.8-PRIa-HCV33-myc were tested for the presence of anti-RR6 binding activity using ELISA. 12 of the tested samples showed low levels of RR6 binding activity. Detailed analysis of the anti-RR6 binding activity present using showed that HCV33-myc was expressed as 0.0002-0.00074% of total protein present (FIG. 9 and Table 2).

### Example 8

### Expression of HCV33 Containing the ER Retention Signal SEKDEL

Small samples of the leaves of 22, 23 and 12 individual Tobacco plants transformed with the expression vectors pPV.8-no leader-HCV33-myc-SEKDEL, pPV.8-PRIa-HCV33-myc-SEKDEL, pPV.8-GBSS-HCV33-myc-SEKDEL respectively were tested for the presence of anti-RR6 binding activity using ELISA. None, 16 and 3 of the tested samples respectively, showed medium to high levels of RR6 binding activity. Detailed analysis of the anti-RR6 binding activity present in these clones using SDS-PAGE/Western blot analysis, ELISA and total protein concentration measurement showed that HCV33-myc was expressed as 0, 0.4 and 0.05% of total protein present respectively (FIG. 10 and Table 2). Localisation of the HCV33 material, which was directed to the secretory pathway by using the PRIa leader sequence, in the ER was confirmed using immuno-histology studies (FIG. 11).

### Example 9

### Effect of Neutralising HCV Expression on SBE Activity in Tobacco Plants

Samples of the leaves of 30 individual tobacco plants transformed with expression vector pSJ.35-CERV-GBSS-Clone46-His6-myc were assayed for SBE activity. Compared to the effect of the HCV33 anti-RR6 antibody in 30 control plants transformed with expression vector pSJ.35-CERV-GBSS-HCV33-myc, the specific anti-potato SBEII antibody Clone46 significantly reduced the SBE activity in the tobacco plants (FIG. 13), indicating that the antibody has neutralised the enzyme activity.

### Example 10

### Effect of Neutralising HCV Expression on GUS Activity in Tobacco Leaves

A tobacco line already transformed with expression vector pCJ.102 and shown to contain GUS activity in leaf tissue was retransformed with the expression vector pSJ35-CERV-Clone18-His6-myc-SEKDEL. Samples from 27 individual transformed plants were assayed for GUS activity in comparison to control pCJ.102 tobacco lines that had not been retransformed. A graphical representation of the results from measuring GUS activity in control lines and in lines containing Clone 18 is shown in FIG. 16. A difference between the two sets of data is clearly evident, indicating that the presence of the anti-GUS antibody has decreased GUS activity.

### Example 11

### Effect of Neutralising HCV Expression on GUS Activity in Tobacco Pollen

The tobacco line lat52-GUS, containing the pollen specific promoter lat52 driving GUS in a single copy homozygous state, was obtained as a gift from Dr. David Twell (Botany Department, University of Leicester, UK) . This line was retransformed with expression vector pSJ.35-Lat52-Clone18-His6-myc-SEKDEL. Control lines were obtained by transforming lat52-GUS tobacco with pSJ.35-Lat52-HCV33-myc-SEKDEL. Pollen from 20 lines of each set of transformed tobacco were tested by histochemical staining for GUS activity. Scanned images showing the staining pattern obtained in a control line and a line containing anti-GUS VHH clone 18 are shown in FIG. 17. It can quite clearly be seen that GUS activity has been ‘knocked-out’ by the presence of anti-GUS antibody. Half the pollen retains GUS activity (shown by blue staining) due to segregation of the transgene at meiosis.

### Example 12

### Expression of Whole Heavy Chain Immunoglobulins in Plants

Construction of HCV33-IgG2b Expression Vectors

pPV.5LN-HCV33-IgG2b

A cDNA clone IgG2b (Vu et. al., Mol. Imm., 34, 16-17, 1997) was amplified by PCR using oligonucleotides 304 (Seq. ID. No. 58) and 305 (Seq. ID. No. 59), 306 (Seq. ID. No. 60) and 310 (Seq. ID. NO. 61) (Table 1). The amplified fragments were digested with BstEII/HincII and HincII/EcoRI respectively and inserted into NcoI/MunI opened vector pPV.5LN together with the NcoI/BstEII HCV33 fragment from vector pPV.5LN-HCV33-myc, yielding the vector pPV.5-HCV33-IgG2b. The DNA sequence of the amplified fragment was verified by sequencing with primers Seq. ID. No. 19, 53, 58, 60 and 62 (Table 1).

The predicted amino acid sequence contained two mutations at amino acid numbers 167 and 369 (FIG. 18) as compared to the published sequence by Woolven et. al., Immunogen., 50, 98-101, 1999. Oligonucleotides 314 (Seq. ID. No. 63) and 307 (Seq. ID. No. 64) (Table 1) were designed to change the mutations at amino acids 167 (proline) and 369 (Stop codon) to a serine and lysine respectively.

The HCV33-IgG2b clone was then amplified by PCR using oligonucleotides 314 and 307 (Table 1). The amplified fragment was digested with SacI/EcoRI and inserted into HindIII/MunI opened vector pPV.5LN together with the HindIII/SacI fragment from vector pPV.5-HCV33-IgG2b, yielding the vector pPV.5LN-HCV33-IgG2b. The DNA sequence of the amplified fragment was verified by sequencing with primers Seq. ID. No. No. 19, 53, 58, 60 and 62 (Table 1) (FIG. 18).

pPV.5LN-PR1a-HCV33-IgG2b

The BstEII/EcoRI HCV33-IgG2b fragment from vector pPV.5LN-HCV33-IgG2b was inserted into HindIII/EcoRI opened pPV.5LN together with the HindIII/BstEII promoter-PR1a-HCV33 fragment from vector pPV.5LN-PR1a-HCV33-myc.

pPV.5LN-GBSS-HCV33-IgG2b

The BstEII/EcoRI HCV33-IgG2b fragment from vector pPV.5LN-HCV33-IgG2b was inserted into HindIII/EcoRI opened pPV.5LN together with the HindIII/BstEII promoter-GBSS-HCV33 fragment from vector pPV.5LN-GBSS-HCV33-myc.

pPV.5LN-PR1a-HCV33-IgG2b-SEKDEL

Vector pPV.5LN-HCV33-IgG2b was amplified by PCR using oligonucleotides 306 and 308 (Seq. ID. No. 65) (Table 1; to generate 3′ SEKDEL endoplasmic retention signal). The amplified fragment was digested with HincII/EcoRI and inserted into HindIII/EcoRI opened pPV.5LN together with the HindIII/HincII fragment from vector pPV.5LN-PR1a-HCV33-IgG2b. The DNA sequence of the amplified fragment was the verified by sequencing with primers Seq. ID. No. 19, 63 (Table 1)(FIG. 19).

pPV.8 2×35S Plant Transformation Vectors

The HindIII/EcoRI inserts from vectors pPV.5LN-HCV33-IgG2b, pPV.5LN-PR1a-HCV33-IgG2b, pPV.5LN-GBSS-HCV33-IgG2b and pPV.5LN-PR1a-HCV33-IgG2b-SEKDEL were inserted into HindIII/EcoRI vector fragment of pSJ.34, yielding pPV.8-HCV33-IgG2b, pPV.8-PR1a-HCV33-IgG2b, pPV.8-GBSS-HCV33-IgG2b and pPV.8-PR1a-HCV33-IgG2b-SEKDEL respectively.

Detection of HCV33-IgG2b Binding Activity

As for RR6 binding activity, except:

Purified and quantified HCV33-IgG2b from tobacco plants was used as the standard.

100 μl/well of 1:2,000 dilution of rabbit anti-llama IgG was used as the primary detection antibody.

100 μl/well of 1 ug/mL goat anti-rabbit antibody conjugated alkaline phosphatase was used as the secondary detection antibody.

Expression of HCV33-IgG2b Antigen Binding Activity in the Cytoplasm

Small samples of the leaves of 20 individual Tobacco plants transformed with the expression vector pPV.8-HCV33-IgG2b were tested for the presence of anti-RR6 binding activity using ELISA. 10 of the tested samples showed low levels of RR6 binding activity. Detailed analysis of the anti-RR6 binding activity showed that HCV33-IgG2b was expressed as 0.011-0.031% of total soluble protein present (FIG. 20).

Three plants chosen at random were selfed and allowed to set seed, the next generation was germinated and after 6 weeks small samples of leaf, stem and root were tested for the presence of anti-RR6 binding activity using ELISA. Two of the tested plants showed increased levels of RR6 binding activity as compared to the original analysis. Detailed analysis of the anti-RR6 binding activity showed that HCV33-IgG2b was expressed at its highest in stem tissues at a maximum level of 1.07% total protein present (FIG. 21).

Expression of HCV33-IgG2b Antigen Binding Activity in the Plastids

Small samples of the leaves of 20 individual Tobacco plants transformed with the expression vector pPV.8-GBSS-HCV33-IgG2b were tested for the presence of anti-RR6 binding activity using ELISA. 10 of the tested samples showed low levels of RR6 binding activity. Detailed analysis of the anti-RR6 binding activity showed that HCV33-IgG2b was expressed as 0.034-0.050% of total soluble protein present (FIG. 22).

Three plants chosen at random were selfed and allowed to set seed, the next generation was germinated and after 6 weeks small samples of leaf, stem and root were tested for the presence of anti-RR6 binding activity using ELISA. All of the tested samples showed increased levels of RR6 binding activity as compared to the original analysis. Detailed analysis of the anti-RR6 binding activity showed that HCV33-IgG2b was expressed at its highest in leaf tissues at a maximum level of 9.26% total soluble protein present (FIG. 23).

Expression of HCV33-IgG2b Antigen Binding Activity in the Apoplastic Space

Small samples of the leaves of 20 individual Tobacco plants transformed with the expression vector pPV.8-PR1a-HCV33-IgG2b were tested for the presence of anti-RR6 binding activity using ELISA. 17 of the tested samples showed significant levels of RR6 binding activity. Detailed analysis of the anti-RR6 binding activity showed that HCV33-IgG2b was expressed as 0.021-0.157% of total soluble protein present (FIG. 24).

Three plants chosen at random were selfed and allowed to set seed, the next generation was germinated and after 6 weeks small samples of leaf, stem and root were tested for the presence of anti-RR6 binding activity using ELISA. All of the tested samples showed increased levels of RR6 binding activity as compared to the original analysis. Detailed analysis of the anti-RR6 binding activity showed that HCV33-IgG2b was expressed at very high levels in all tissues tested, with the highest level found in leaf at a maximum of 13% total soluble protein present (FIG. 25).

Expression of HCV33-IgG2b Antigen Binding Activity in the Endoplasmic Reticulum

Small samples of the leaves of 20 individual Tobacco plants transformed with the expression vector pPV.8-HCV33-IgG2b were tested for the presence of anti-RR6 binding activity using ELISA. 13 of the tested samples showed significant levels of RR6 binding activity. Detailed analysis of the anti-RR6 binding activity present using showed that HCV33-IgG2b was expressed as 0.02-0.165% of total soluble protein present (FIG. 26).

Three plants chosen at random were selfed and allowed to set seed, the next generation was germinated and after 6 weeks small samples of leaf, stem and root were tested for the presence of anti-RR6 binding activity using ELISA. All of the tested samples showed increased levels of RR6 binding activity as compared to the original analysis. Detailed analysis of the anti-RR6 binding activity showed that HCV33-IgG2b was expressed at very high levels in all tissues tested, with the highest level found in leaf at a maximum of 16.3% of total soluble protein present (FIG. 27).

### Example 13

### Expression of Anti-SBEII (Clone68) Antigen Binding Activity in Tobacco Directed to the Plastids

Construction of CERV-GBSS-Clone68-myc Expression Vectors

pPV.7LN-GBSS-Clone68-His6-myc

The Clone68-His6-myc sequence was amplified by PCR using oligonucleotides PCR.288 (Seq. ID. No. 47) and JWHCV2 (Seq. ID. No. 48) (Table 1) from pHEN.Clone68-His6-myc. The PstI/MunI fragment was inserted into the NcoI/MunI opened vector pPV.7LN together with a 252 bp NcoI/PstI fragment encoding the GBSS leader sequence (as described for pPV.5LN-GBSS-HCV33-myc) allowing the in-frame fusion with the 5′ framework-1 sequences of the HCV fragment to create pPV.7LN-GBSS-Clone68-His6-myc. The DNA sequence of clone 68 was then verified with sequencing primers, Seq. ID. No. 19,49 (Table 1)(FIG. 28).

pSJ.35-CERV Plant Transformation Vector

The HindIII/EcoRI inserts from vector pPV.7LN-GBSS-clone68-His6-myc were inserted into HindIII/EcoRI vector fragment of pSJ.35, yielding vector pSJ.35-CERV-GBSS-clone68-His6-myc.

Detection of SBEII Binding Activity

This was performed as described in example 3 for anti-SBEII binding activity, except that purified and quantified clone68-His6-myc from E.coli was used as the standard.

Expression of Anti-SBEII Antigen Binding Activity in the Plastids

High levels of expression were obtained with another HCV antibody with anti-SBEII antigen binding specificity. Small samples of the leaves of 30 individual tobacco plants transformed with the expression vector pSJ.35-CERV-GBSS-Clone68-His6-myc were tested for the presence of anti-SBEII binding activity using ELISA. 26 of the tested plants expressed levels of SBEII-binding activity above the standards with expression levels at a minimum of 0.3% total soluble protein (FIG. 29).

TABLE 1

DNA sequence of the synthetic oligonucleotides used in
the examples.

| | SEQ. ID. | |
|---|---|---|
| Code | No. | Nucleotide Sequence |
| PCR.624 | 12 | ACCTGCGATCGCAAGCTTGGTACCGGCAATTCTCTAGA |
| PCR.625 | 13 | AATTTCTAGAGAATTCCCGGTACCAAGCTTGCTTGCGATCGC |
| PCR.626 | 14 | TCGACCCATGGCCCGCTAGCCAATTGGACCT |
| PCR.627 | 15 | CCAATTGGCTAGCGGGCCATGGG |
| PCR.640 | 16 | CCACCCACGAGGGAACATCGTG |
| PCR.641 | 17 | CAATTCCCATGGTTTACACTCGAGGTCCTCTCCAAATGA |
| 172 | 19 | CCGGCAACAGGATTCAATCTT |
| PCR.293 | 20 | AGCTCCATGGGATTTGTTCTCTTTTCACAATTGCCTTCAT |
| PCR.294 | 21 | TTCTTCTTGTCTCTACACTTCTCTTATTCCTAGTA |
| PCR.295 | 22 | ATATCCCACTCTTCCCGTGCCCAGGTCCAGCTGCA |
| PCR.296 | 23 | GCTGCACCTGGGCACGCCAAGAGTGGGATATTACTAGGAATAAGAGAA |
| PCR.297 | 24 | GTGTAGAGACAAGAAGAAATGAAGGCAATTGTGAAAAGAGAACAAATCCC |
| | | ATGG |
| 152 | 25 | GGAAACAGCTATGACCATGATTAC |
| 191 | 26 | TTTCCCAGTCACGACGTTGT |
| 132 | 29 | GACATCCCATGGCAAGCATCA |
| 133 | 30 | AAGCTTGTTAACAGCCCTTAA |
| 134 | 31 | AGGGCTGTTAACAAACTTGAT |
| 136 | 32 | AGACTGCTGCAGCTGCACCTGCTTTCCACAAACAATCCTAGCTG |
| 197 | 33 | AATTAACCCTCACTAAAGG |
| PCR.652 | 36 | CATGCAGGTGCAGCTGCA |
| PCR.653 | 37 | GCTGCACCTG |
| PCR.300 | 38 | CGCAAGACCCTTCCTCTATATAAG |
| PCR.690 | 39 | GAGCTCGAATTCTTATTATAGCTCATCTTTCTCTGAATTCAGATCCTCTT |
| | | CTGAGATGAG |
| CERV1 | 41 | ATCCTCAACTTCCAATCAGA |
| CERV2 | 42 | TTCTTGAGAGATACCTTGA |
| PCR.628 | 43 | GATCCCATGGCCCGCTAGCCAATTGGAGCT |
| PCR.629 | 44 | CCAATTCGCTACCGGCCCATCG |
| PCR.645 | 45 | GATCCACCTCGAGTGTAAAOCATGGCCCC |
| PCR.646 | 46 | CTAGCGGGCCATGGTTTACACTCGAGGTG |
| PCR.288 | 47 | ATTGCCTACGGCAGCCGCTG |
| JWHCV2 | 48 | TCCAACCAATTGTTACTATGCGGCCCCATTCAGATCCTCTTCTGAG |
| | | ATGAG |
| 164 | 49 | GTCTGTCTAAAGTAAAGTAGATGCG |
| JWHCV1 | 50 | TCCAACCAATTGTTATCATAGCTCATCTTTCTCACTATTCAGATCC |
| | | TCTTCTGAGATGAG |
| 167 | 51 | AGTCCCCCATGGTACGTCCTGTAGAAACC |
| 168 | 52 | CGTTTTCGTCGGTAATCACCATTCC |
| 161 | 53 | CGCAAGACCCTTCCTTTATATAAG |
| 304 | 58 | AGGGGACCCAGGTCACCGTCTGCTCAGAACC |
| 305 | 59 | GAGCTTTGTTGTTGACCTTGCATTTGAACTCTTTCCC |
| 306 | 60 | CAAATGCAAGGTCAACAACAAAGCTC |
| 310 | 61 | TTCGAGCTCGAATTCTTATTATTTACCCGAAGACTGGGTGAT |
| 303 | 62 | CTGAGGAGACGGTGACCTGGGTCCCCT |
| 314 | 63 | AGCCCCTGAGCTCCTGGGAGGGCCCTCAGTCTTCATCTTCCCCCCG |
| 307 | 64 | TTCGAGCTCGAATTCTTATTATTTACCCGAAGACTGGGTGATGGATTTCT |
| | | GGGTGTAGTGG |
| 308 | 65 | TTCGAGCTCGAATTCTTATTATAGCTCATCTTTCTCAGATTTACCCGAAG |
| | | ACTGGGTGATGGATTTCTGGGTGTAGTGG |

TABLE 2

Summary of HCV expression levels in tobacco
(unless otherwise stated) detected. Maximum
observed expression levels are expressed as
percentage of active antibody in the total amount
of soluble protein extracted.

| | | |
|---|---|---|
| | | Expression |
| Construct | Compartment | Level |

| | | |
|---|---|---|
| pPV.8-no leader-HCV33-myc | Cytosol | 0.002% |
| pPV.8-GBSS-HCV33-myc | Chloroplast | 1.0% |
| pPV.8-PRIa-HCV33-myc | Apoplast | 0.00074% |
| pPV.8-no leader-HCV33-myc- | Cytosol | >0.001% |
| His6-myc | | |
| pPV.8-GBSS-HCV33-myc-KDEL | Chloroplast | 0.05% |
| pPV.8-PRIa-HCV33-myc-KDEL | ER | 0.4% |
| pPV.8-GBSS-HCV33-myc | Plastid | 0.2% |
| | (Tomato fruit | |
| | columella) | |
| pSJ.35-CERV-GBSS-Clonel- | Chloroplast | 0.2% |
| His6-myc | | |

1. A method for modifying a plant to produce an antibody or an active fragment or derivative thereof, or a protein functionally equivalent thereto, in a desired cellular compartment comprising introducing into a plant a DNA sequence encoding a heavy chain immunoglobulin or an active fragment or derivative thereof, or a sequence encoding a protein functionally equivalent thereto, said DNA sequence being operably linked to one or more promoters and provided, as appropriate, with an additional sequence encoding a peptide sequence capable of targeting said antibody or fragment or derivative thereof to said desired cellular compartment.

2. A method according to claim 1 wherein the DNA sequence encoding the heavy chain immunoglobulin or fragment or derivative thereof is obtainable from camelids.

3. A method according to claim 1 or claim 2 wherein the plant is selected from tobacco, pea, potato, spinach, tomato or tea.

4. A method according to claim 1 wherein the heavy chain immunoglobulin or active fragment or derivative thereof binds to a protein present in the plant.

5. A method according to claim 1 wherein the heavy chain immunoglobulin or active fragment or derivative thereof binds to a plant or animal pathogen.

6. A method according to claim 1 wherein the heavy chain immunoglobulin or active fragment or derivative thereof binds to a plant hormone or metabolite.

7. A plant prepared according to the method of claim 1.

8. A modified plant having, in a desired cellular compartment, enhanced levels of heavy chain immunoglobulins or active fragments or derivatives thereof or proteins functionally equivalent thereto compared to equivalent but unmodified plants.

9. Seeds, fruits, progeny and hybrids of a plant according to claim 7 or 8.

10. A food product comprising a plant according to claim 7 or 8.

11. A method for increasing pathogen resistance in a plant comprising introducing into said plant a DNA sequence encoding a heavy chain immunoglobulin which binds to a plant or animal pathogen, or an active fragment or derivative thereof or one or more sequences encoding a protein functionally equivalent thereto, according to the method of claim 1.

12. A method for modulating plant metabolism comprising introducing into said plant a DNA sequence encoding a heavy chain immunoglobulin which binds to a protein present in said plant or an active fragment or derivative thereof or one or more sequences encoding a protein functionally equivalent thereto according to the method of claim 1.

13. A method for preparing a heavy chain immunoglobulin or an active fragment or derivative thereof comprising the steps of:
(i) modifying a plant according to the method of claim 1, and
(ii) extracting from said modified plant the heavy chain immunoglobulin or active fragment or derivative thereof produced therein.


SEQUENCE LISTING (67 sequences)

SEQ ID NO: 1 - DNA, 440 bp, Artificial Sequence
  VHH with peptide linker
caggtgcagctgcaggagtcagggggaggattggtgcaggctgggggctctctgagactctcctgtgcagcctcgggacgcgccaccagtggtcatggtcactatggtatgggctggttccgccaggttccagggaaggagcgtgagtttgtcgcagctattaggtggagtggtaaagagacatggtataaagactccgtgaagggccgattcaccatctccagagataacgccaagactacggtttatctgcaaatgaacagcctgaaacctgaagatacggccgtttattattgtgccgctcgaccggtccgcgtggatgatatttccctgccggttgggtttgactactggggccaggggacccaggtcaccgtctcctcagaacaaaaactcatctcagaagaggatctgaattaataagggctaagctcgaattc

SEQ ID NO: 2 - protein, 139 aa, Artificial Sequence
  VHH with peptide linker
QVQLQESGGGLVQAGGSLRLSCAASGRATSGHGHYGMGWFRQVPGKEREFVAAIRWSGKETWYKDSVKGRFTISRDNAKTTVYLQMNSLKPEDTAVYYCAARPVRVDDISLPVGFDYWGQGTQVTVSSEQKLISEEDLN

SEQ ID NO: 3 - protein, 11 aa, Artificial Sequence
  myc linker
EQKLISEEDLN

SEQ ID NO: 4 - DNA, 471 bp, Artificial Sequence
  VHH with linker
caggtgcagctgcagcagtcagggggaggcttggtgcaggctggggggtctctgagactctcctgtgtagcttctgaaagcagcttcagcaacaatcacatgggctggtaccgccgggctccagggaaccagcgcgagctggtcgcaactattagtcctggtggtagcacacactatgtagactccgtgaagggccgattcaccatctcccgagacaacgccaagaacacagtgtatctacaaatggacagcctgaaaccagaggacacggccgtctattactgtgctgccaaggggagggggctgcaggctatgcagtactggggccaggggaccctggtcaccgtctcctcagcgcaccacagcgaagaccccagctccgcggccgcccatcaccatcaccatcacggggccgcagaacaaaaactcatctcagaagaggatctgaatggggccgcatagtaacaattg

SEQ ID NO: 5 - protein, 153 aa, Artificial Sequence
  VHH with linker
QVQLQQSGGGLVQAGGSLRLSCVASESSFSNNHMGWYRRAPGNQRELVATISPGGSTHYVDSVKGRFTISRDNAKNTVYLQMDSLKPEDTAVYYCAAKGRGLQAMQYWGQGTLVTVSSAHHSEDPSSAAAHHHHHHGAAEQKLISEEDLNGAA

SEQ ID NO: 6 - DNA, 468 bp, Artificial Sequence
  VHH with linker
caggtgcagctgcaggagtctgggggaggcctggtgcaggctggggggtctctgagactctcctgtgtagcctctggaaacaccttcagtatcatagctatggcctggtaccgccaggctccagggaagcagcgcgaggtggtcgcaagtattaatagtattggcagcacaaattatgcagactccgtgaaggggcgattcaccatctccagagacaacgccaagaacacagtgtatctgcaaatgagcagcctgaaacctgaggacacggccgtctattactgtgctgccggtaatttgctggttaagaggccttactggggccaggggaccctggtcaccgtctcctcagaacccaagacaccaaaaccacaaccagcggccgcccatcaccatcaccatcacggggccgcagaacaaaaactcatctcagaagaggatctgaatggggccgcatagtaacaattg

SEQ ID NO: 7 - protein, 152 aa, Artificial Sequence
  VHH with linker
QVQLQESGGGLVQAGGSLRLSCVASGNTFSIIAMAWYRQAPGKQREVVASINSIGSTNYADSVKGRFTISRDNAKNTVYLQMSSLKPEDTAVYYCAAGNLLVKRPYWGQGTLVTVSSEPKTPKPQPAAAHHHHHHGAAEQKLISEEDLNGAA

SEQ ID NO: 8 - DNA, 462 bp, Artificial Sequence
  VHH with linker
accatggcccaggtgaaactgcagcagtctgggggaggattggtgcaggctgggggccctctgaggctctcctgtgcagcctctggacgcaccttcagtaactatgccgtgggctggttccgccaggctccagggaaggagcgtgagtttgtcgctgctattagccgtgatggtgggcgcacatactatgcggactccgtgaagggccgattcgccgtctccagagactacgccgagaacacggtgtatctgcaaatgaacagcctgaaacctgaggacacggccgtttattactgtaacacaagggcctactggggccaggggacccaggtcaccgtctcctcagcgcaccacagcgaagaccccagctccgcggccgcccatcaccatcaccatcacggggccgcagaacaaaaactcatctcagaagaggatctgaatggggccgcatagtaacaattg

SEQ ID NO: 9 - protein, 150 aa, Artificial Sequence
  VHH with linker
TMAQVKLQQSGGGLVQAGGPLRLSCAASGRTFSNYAVGWFRQAPGKEREFVAAISRDGGRTYYADSVKGRFAVSRDYAENTVYLQMNSLKPEDTAVYYCNTRAYWGQGTQVTVSSAHHSEDPSSAAAHHHHHHGAAEQKLISEEDLNGAA

SEQ ID NO: 10 - DNA, 471 bp, Artificial Sequence
  VHH with linker
accatggcccaggtgaaactgcagcagtctgggggaggattggtgcaggctgggggccctctgaggctctcctgtgcagcctctggacgcaccttcagtaactatgccgtgggctggttccgccaggctccagggaaggagcgtgagtttgtcgctgctattagccgtgatggtgggcgcacatactatgcggactccgtgaagggccgattcgccgtctccagagactacgccgagaacacggtgtatctgcaaatgaacagcctgaaacctgaggacacggccgtttattactgtaacacaagggcctactggggccaggggacccaggtcaccgtctcctcagcgcaccacagcgaagaccccagctccgcggccgcccatcaccatcaccatcacggggccgcagaacaaaaactcatctcagaagaggatctgaatagtgagaaagatgagctatgataacaattg

SEQ ID NO: 11 - protein, 153 aa, Artificial Sequence
  VHH with linker
TMAQVKLQQSGGGLVQAGGPLRLSCAASGRTFSNYAVGWFRQAPGKEREFVAAISRDGGRTYYADSVKGRFAVSRDYAENTVYLQMNSLKPEDTAVYYCNTRAYWGQGTQVTVSSAHHSEDPSSAAAHHHHHHGAAEQKLISEEDLNSEKDEL

SEQ ID NO: 12 - DNA, 38 bp, Artificial Sequence
  synthetic insert
agctgcgatcgcaagcttggtaccgggaattctctaga

SEQ ID NO: 13 - DNA, 42 bp, Artificial Sequence
  synthetic insert
aatttctagagaattcccggtaccaagcttgcttgcgatcgc

SEQ ID NO: 14 - DNA, 31 bp, Artificial Sequence
  synthetic insert
tcgacccatggcccgctagccaattggagct

SEQ ID NO: 15 - DNA, 23 bp, Artificial Sequence
  synthetic insert
ccaattggctagcgggccatggg

SEQ ID NO: 16 - DNA, 22 bp, Artificial Sequence
  primer
ccacccacgagggaacatcgtg

SEQ ID NO: 17 - DNA, 39 bp, Artificial Sequence
  primer
gaattcccatggtttacactcgaggtcctctccaaatga

SEQ ID NO: 18 - DNA, 189 bp, Artificial Sequence
  PCR product
ccacccacgagggaacatcgtggaaaaagaagacgttccaaccacgtcttcaaagcaagtggattgatgtgatatctccactgacgtaagggatgacgcacaatcccactatccttcgcaagacccttcctttatataaggaagttcatttcatttggagaggacctcgagtgtaaaccatgggaattc

SEQ ID NO: 19 - DNA, 21 bp, Artificial Sequence
  sequencing primer
ccggcaacaggattcaatctt

SEQ ID NO: 20 - DNA, 40 bp, Artificial Sequence
  synthetic insert
agctccatgggatttgttctcttttcacaattgccttcat

SEQ ID NO: 21 - DNA, 35 bp, Artificial Sequence
  synthetic insert
ttcttcttgtctctacacttctcttattcctagta

SEQ ID NO: 22 - DNA, 35 bp, Artificial Sequence
  synthetic insert
atatcccactcttgccgtgcccaggtgcagctgca

SEQ ID NO: 23 - DNA, 48 bp, Artificial Sequence
  synthetic insert
gctgcacctgggcacggcaagagtgggatattactaggaataagagaa

SEQ ID NO: 24 - DNA, 54 bp, Artificial Sequence
  synthetic insert
gtgtagagacaagaagaaatgaaggcaattgtgaaaagagaacaaatcccatgg

SEQ ID NO: 25 - DNA, 24 bp, Artificial Sequence
  synthetic insert
ggaaacagctatgaccatgattac

SEQ ID NO: 26 - DNA, 20 bp, Artificial Sequence
  synthetic insert
tttcccagtcacgacgttgt

SEQ ID NO: 27 - DNA, 107 bp, Artificial Sequence
  synthetic fragment encoding PRIa leader
ccatgggatttgttctcttttcacaattgccttcatttcttcttgtctctacacttctcttattcctagtaatatcccactcttgccgtgcccaggtgcagctgcag

SEQ ID NO: 28 - protein, 34 aa, Artificial Sequence
  synthetic fragment encoding PRIa leader
MGFVLFSQLPSFLLVSTLLLFLVISHSCRAQVQL

SEQ ID NO: 29 - DNA, 21 bp, Artificial Sequence
  PCR primer
gacatcccatggcaagcatca

SEQ ID NO: 30 - DNA, 21 bp, Artificial Sequence
  PCR primer
aagcttgttaacagcccttaa

SEQ ID NO: 31 - DNA, 21 bp, Artificial Sequence
  PCR primer
agggctgttaacaaacttgat

SEQ ID NO: 32 - DNA, 44 bp, Artificial Sequence
  PCR primer
agactgctgcagctgcacctgctttccacaaacaatggtagctg

SEQ ID NO: 33 - DNA, 19 bp, Artificial Sequence
  sequencing primer
aattaaccctcactaaagg

SEQ ID NO: 34 - DNA, 254 bp, Artificial Sequence
  GBSS leader
ccatggcaagcatcacagcttcacaccactttgtgtcaagaagccaaacttcactagacaccaaatcaaccttgtcacagataggactcaggaaccatactctgactcacaatggtttaagggctgttaacaaacttgatgggctccaatcaagaactaatactaaggtaacacccaagatggcatccagaactgagaccaagagacctggatgctcagctaccattgtttgtggaaaacaggtgcagctgcag

SEQ ID NO: 35 - protein, 84 aa, Artificial Sequence
  GBSS leader
MASITASHHFVSRSQTSLDTKSTLSQIGLRNHTLTHNGLRAVNKLDGLQSRTNTKVTPKMASRTETKRPGCSATIVCGKQVQLQ

SEQ ID NO: 36 - DNA, 18 bp, Artificial Sequence
  synthetic insert
catgcaggtgcagctgca

SEQ ID NO: 37 - DNA, 10 bp, Artificial Sequence
  synthetic insert
gctgcacctg

SEQ ID NO: 38 - DNA, 24 bp, Artificial Sequence
  PCR primer
cgcaagacccttcctctatataag

SEQ ID NO: 39 - DNA, 60 bp, Artificial Sequence
  PCR primer
gagctcgaattcttattatagctcatctttctctgaattcagatcctcttctgagatgag

SEQ ID NO: 40 - protein, 26 aa, Artificial Sequence
  linker
TVTVSSGGGGSGGGGSGGGGSDIELT

SEQ ID NO: 41 - DNA, 20 bp, Artificial Sequence
  PCR primer
atcctcaacttccaatcaga

SEQ ID NO: 42 - DNA, 19 bp, Artificial Sequence
  PCR primer
ttcttgagagatagcttga

SEQ ID NO: 43 - DNA, 30 bp, Artificial Sequence
  synthetic insert
gatcccatggcccgctagccaattggagct

SEQ ID NO: 44 - DNA, 22 bp, Artificial Sequence
  synthetic insert
ccaattggctagcgggccatgg

SEQ ID NO: 45 - DNA, 29 bp, Artificial Sequence
  synthetic insert
gatccacctcgagtgtaaaccatggcccg

SEQ ID NO: 46 - DNA, 29 bp, Artificial Sequence
  synthetic insert
ctagcgggccatggtttacactcgaggtg

SEQ ID NO: 47 - DNA, 20 bp, Artificial Sequence
  PCR primer
attgcctacggcagccgctg

SEQ ID NO: 48 - DNA, 51 bp, Artificial Sequence
  PCR primer
tccaaccaattgttactatgcggccccattcagatcctcttctgagatgag

SEQ ID NO: 49 - DNA, 25 bp, Artificial Sequence
  sequencing primer
gtctgtctaaagtaaagtagatgcg

SEQ ID NO: 50 - DNA, 60 bp, Artificial Sequence
  PCR primer
tccaaccaattgttatcatagctcatctttctcactattcagatcctcttctgagatgag

SEQ ID NO: 51 - DNA, 29 bp, Artificial Sequence
  PCR primer
agtcccccatggtacgtcctgtagaaacc

SEQ ID NO: 52 - DNA, 25 bp, Artificial Sequence
  PCR primer
cgttttcgtcggtaatcaccattcc

SEQ ID NO: 53 - DNA, 24 bp, Artificial Sequence
  sequencing primer
cgcaagacccttcctttatataag

SEQ ID NO: 54 - DNA, 1154 bp, Artificial Sequence
  HCV33-hinge-CH2-CH3
ccatggaggtgcagctgcaggagtcagggggaggattggtgcaggctgggggctctctgagactctcctgtgcagcctcgggacgcgccaccagtggtcatggtcactatggtatgggctggttccgccaggttccagggaaggagcgtgagtttgtcgcagctattaggtggagtggtaaagagacatggtataaagactccgtgaagggccgattcaccatctccagagataacgccaagactacggtttatctgcaaatgaacagcctgaaacctgaagatacggccgtttattattgtgccgctcgaccggtccgcgtggatgatatttccctgccggttgggtttgactactggggccaggggacccaggtcaccgtctcctcagaacccaagacaccaaaaccacaaccacaaccacaaccacaaccacaacccaatcctacaacagaatccaagtgtcccaaatgtccagcccctgagctcctgggagggccctcagtcttcatcttccccccgaaacccaaggacgtcctctccatttctgggaggcccgaggtcacgtgcgttgtggtagacgtgggccaggaagaccccgaggtcagtttcaactggtacattgatggcgcagaggtgcgaacggccaacacgaggccaaaagaggaacagttcaacagcacgtaccgcgtggtcagcgtcctgcccatccagcaccaggactggctgacggggaaagagttcaaatgcaaggtcaacaacaaagctctcccggcccccatcgagaagaccatctccaaggccaaagggcagacccgggagccgcaggtgtacgccctggccccacaccgggaagagctggccaaggacaccgtgagcgtaacctgcctggtcaaaggcttctacccacctgatatcaacgttgagtggcagaggaacggtcagccggagtcagagggcacctacgccaccacgccaccccagctggacaacgacgggacctacttcctctacagcaagctctcggtgggaaagaacacgtggcagcggggagaaaccttcacctgtgtggtgatgcacgaggccctgcacaaccactacacccagaaatccatcacccagtcttcgggtaaataataagaattcgagctcgaa

SEQ ID NO: 55 - protein, 377 aa, Artificial Sequence
  HCV33-hinge-CH2-CH3
MEVQLQESGGGLVQAGGSLRLSCAASGRATSGHGHYGMGWFRQVPGKEREFVAAIRWSGKETWYKDSVKGRFTISRDNAKTTVYLQMNSLKPEDTAVYYCAARPVRVDDISLPVGFDYWGQGTQVTVSSEPKTPKPQPQPQPQPQPNPTTESKCPKCPAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYALAPHREELAKDTVSVTCLVKGFYPPDINVEWQRNGQPESEGTYATTPPQLDNDGTYFLYSKLSVGKNTWQRGETFTCVVMHEALHNHYTQKSITQSSGK

SEQ ID NO: 56 - DNA, 1172 bp, Artificial Sequence
  hinge-HCV33-CH2-CH3-SEKDEL
ccatggaggtgcagctgcaggagtcagggggaggattggtgcaggctgggggctctctgagactctcctgtgcagcctcgggacgcgccaccagtggtcatggtcactatggtatgggctggttccgccaggttccagggaaggagcgtgagtttgtcgcagctattaggtggagtggtaaagagacatggtataaagactccgtgaagggccgattcaccatctccagagataacgccaagactacggtttatctgcaaatgaacagcctgaaacctgaagatacggccgtttattattgtgccgctcgaccggtccgcgtggatgatatttccctgccggttgggtttgactactggggccaggggacccaggtcaccgtctcctcagaacccaagacaccaaaaccacaaccacaaccacaaccacaaccacaacccaatcctacaacagaatccaagtgtcccaaatgtccagcccctgagctcctgggagggccctcagtcttcatcttccccccgaaacccaaggacgtcctctccatttctgggaggcccgaggtcacgtgcgttgtggtagacgtgggccaggaagaccccgaggtcagtttcaactggtacattgatggcgcagaggtgcgaacggccaacacgaggccaaaagaggaacagttcaacagcacgtaccgcgtggtcagcgtcctgcccatccagcaccaggactggctgacggggaaagagttcaaatgcaaggtcaacaacaaagctctcccggcccccatcgagaagaccatctccaaggccaaagggcagacccgggagccgcaggtgtacgccctggccccacaccgggaagagctggccaaggacaccgtgagcgtaacctgcctggtcaaaggcttctacccacctgatatcaacgttgagtggcagaggaacggtcagccggagtcagagggcacctacgccaccacgccaccccagctggacaacgacgggacctacttcctctacagcaagctctcggtgggaaagaacacgtggcagcggggagaaaccttcacctgtgtggtgatgcacgaggccctgcacaaccactacacccagaaatccatcacccagtcttcgggtaaatctgagaaagatgagctataataagaattcgagctcgaa

SEQ ID NO: 57 - protein, 383 aa, Artificial Sequence
  hinge-HCV33-CH2-CH3-SEKDEL
MEVQLQESGGGLVQAGGSLRLSCAASGRATSGHGHYGMGWFRQVPGKEREFVAAIRWSGKETWYKDSVKGRFTISRDNAKTTVYLQMNSLKPEDTAVYYCAARPVRVDDISLPVGFDYWGQGTQVTVSSEPKTPKPQPQPQPQPQPNPTTESKCPKCPAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYALAPHREELAKDTVSVTCLVKGFYPPDINVEWQRNGQPESEGTYATTPPQLDNDGTYFLYSKLSVGKNTWQRGETFTCVVMHEALHNHYTQKSITQSSGKSEKDEL

SEQ ID NO: 58 - DNA, 31 bp, Artificial Sequence
  PCR primer
aggggacccaggtcaccgtctcctcagaacc

SEQ ID NO: 59 - DNA, 37 bp, Artificial Sequence
  PCR primer
gagctttgttgttgaccttgcatttgaactctttccc

SEQ ID NO: 60 - DNA, 26 bp, Artificial Sequence
  PCR primer
caaatgcaaggtcaacaacaaagctc

SEQ ID NO: 61 - DNA, 42 bp, Artificial Sequence
  PCR primer
ttcgagctcgaattcttattatttacccgaagactgggtgat

SEQ ID NO: 62 - DNA, 27 bp, Artificial Sequence
  sequencing primer
ctgaggagacggtgacctgggtcccct

SEQ ID NO: 63 - DNA, 46 bp, Artificial Sequence
  PCR primer
agcccctgagctcctgggagggccctcagtcttcatcttccccccg

SEQ ID NO: 64 - DNA, 61 bp, Artificial Sequence
  PCR primer
ttcgagctcgaattcttattatttacccgaagactgggtgatggatttctgggtgtagtgg

SEQ ID NO: 65 - DNA, 79 bp, Artificial Sequence
  PCR primer
ttcgagctcgaattcttattatagctcatctttctcagatttacccgaagactgggtgatggatttctgggtgtagtgg

SEQ ID NO: 66 - DNA, 461 bp, Artificial Sequence
  VHH with linker
ccatggcccaggtgcagctgcaggagtctgggggaggcttggtgcaggctggggggtctctgaggctctcctgtgcagcctctggaagcattttcagacgtccgcatatgggttggttccgccaggctccagggcaggagcgcgagttggtcgcactgatttctgcgggtggtcgtacatggtatgcagactccgtgaagggccgattcaccatctccagagacaacgccaagaacacgctgtatctgcaaatgaacagcctgaaacctgaggacacggccgtttattactgtactgccgggggttcgtactggggccaggggacccaggtcaccgtcgcctcagaacccaagacaccaaaaccacaaccagcggccgcccatcaccatcaccatcacggggccgcagaacaaaaactcatctcagaagaggatctgaatggggccgcatagtaacaattg

SEQ ID NO: 67 - protein, 149 aa, Artificial Sequence
  VHH with linker
MAQVQLQESGGGLVQAGGSLRLSCAASGSIFRRPHMGWFRQAPGQERELVALISAGGRTWYADSVKGRFTISRDNAKNTLYLQMNSLKPEDTAVYYCTAGGSYWGQGTQVTVASEPKTPKPQPAAAHHHHHHGAAEQKLISEEDLNGAA